(19) United States
(12) Patent Application Publication
(10) Pub. No.: US 2024/0076703 A1
(43) Pub. Date: Mar. 7, 2024

(54) S-METHYLTHIORIBOSE KINASE POLYPEPTIDES AND PROCESSES FOR MAKING AND USING S-METHYLTHIORIBOSE KINASE POLYPEPTIDES

(71) Applicant: Merck Sharp & Dohme LLC, Rahway, NJ (US)

(72) Inventors: Tamas Benkovics, Louisville, CO (US); Hsing-I Ho, New Providence, NJ (US); John McIntosh, Brookline, MA (US); Grant S. Murphy, Princeton, NJ (US); Weilan Pan, Downington, PA (US); Deeptak Verma, Feasterville-Trevose, PA (US); Hao Yang, Scotch Plains, NJ (US)

(73) Assignee: Merck Sharp & Dohme LLC, Rahway, NJ (US)

(21) Appl. No.: 18/256,061

(22) PCT Filed: Dec. 9, 2021
(86) PCT No.: PCT/US2021/062503

§ 371 Date: Jun. 6, 2023

Related U.S. Application Data
(60) Provisional application No. 63/278,284, filed on Nov. 11, 2021.
(60) Provisional application No. 63/125,154, filed on Dec. 14, 2020.

(51) Int. Cl. C12P 17/04; C12N 9/12; C12N 15/70

(52) U.S. Cl. CPC C12P 17/04; C12N 9/1205; C12N 15/70; C12Y 207/011; C12N 2800/101

(57) ABSTRACT

The present disclosure provides MTR kinase polypeptides having improved properties as compared to a naturally occurring wild-type MTR kinase polypeptide including the capability of phosphorylating D-ribose and 5′-D-isobutyrylribose to give alpha-D-ribose-1-phosphate and alpha 5′-D-isobutyrylribose-1-phosphate. Also provided are polynucleotides encoding the MTR kinase polypeptides, host cells capable of expressing the MTR kinase polypeptides, and methods of using the MTR kinase polypeptides to synthesize alpha-D-ribose-1-phosphate and alpha 5′-D-isobutyrylribose-1-phosphate.

### FIELD OF THE INVENTION

The present disclosure relates to S-methylthioribose kinase polypeptides, useful in the biocatalytic and synthetic processes. Such enzymes may be particularly useful in synthetic processes that may be used as part of the preparation of ribonucleosides containing 5′-acyl or alkyl groups or of intermediates formed during preparation of such ribonucleosides.

### BACKGROUND OF THE INVENTION

Enzymes are polypeptides that serve to accelerate the chemical reactions of living cells (often by several orders of magnitude). Without enzymes, most biochemical reactions would be too slow to even carry out life processes. Enzymes display great specificity and are not permanently modified by their participation in reactions. Because they are not changed during the reactions, enzymes can be cost effectively used as catalysts for a desired chemical transformation.

S-methylthioribose kinases, also known as MTR kinases, are phosphorylation catalysts agents, a specific class of enzymes that catalyze the selective 1-phosphorylation of S-methyl-5-thioribose. Synthetic applications of these enzymes have not been previously reported in the academic literature. Enzymes belonging to the MTR kinase class are herein shown to be useful for the synthesis of 1-phosphorylated sugars and that can then be converted to the corresponding nucleoside by the action of a nucleoside phosphorylase. MTR kinases may selectively phosphorylate the 1-position of ribose and ribose analogs using ATP. Enzymatic phosphorylation requires participation of a co-factor that can act as a phosphoryl donor, often a nucleotide triphosphate such as ATP.

MTR kinases have been the subject of limited biochemical characterization to date. See, e.g., Kenneth A. Cornell et al., 317 BIOCHEM. J. 285-290 (1996); Toshihiro Nakano et al., 77(5) BIOSCI. BIOTECHNOL. BIOCHEM. 1104-1107 (2013); Agnieszka Sekowska et al., BMC MICROBIOLOGY 2001, 1:15; Andrezej Guranowski, 71 PLANT PHYSIOL. 932-935 (1983); Shao-Yang Ku et al., ACTA CRYST. (2004) D60, 116-119; Margret Sauter et al., 136 PLANT PHYSIOLOGY 4061-6071 (December 2004). Using bioinformatic alignments, it is possible to identify additional putative members of this enzyme family. Herein, the usefulness of these enzymes for use in synthesis is demonstrated.

### SUMMARY OF THE INVENTION

The present disclosure relates to polypeptides that are MTR kinases capable of converting ribose and 5-isobutyrylribose to the corresponding 1-phosphate, particularly as part of nucleotide synthesis. In embodiments, the subject MTR kinases described herein are capable of converting ribose to ribose-1-phosphate or 5-isobutyrylribose to 5-isobutyrylribose-1-phosphate. In particular, the subject MTR kinases described herein may be useful in the preparation of nucleosides, such as uridine 4-oxime 5′-(2-methylpropanoate), and in particular {(2R,3S,4R,5R) -3,4-dihydroxy-5-[(4Z)-4-(hydroxyimino)-2-oxo-3,4dihydropyrimidin-1(2H)-yl]oxolan-2-yl}methyl 2-methylpropanoate. Such nucleosides may be useful as biologically active compounds or as an intermediate for the synthesis of more complex biologically active compounds.

Additional embodiments describe processes for preparing the subject MTR kinases and processes for using the subject MTR kinases.

Other embodiments, aspects and features of the present disclosure are either further described in or will be apparent from the ensuing description, examples and appended claims.

### DETAILED DESCRIPTION OF THE INVENTION

### Definitions

Certain technical and scientific terms are specifically defined below. Unless specifically defined elsewhere in this document, all other technical and scientific terms used herein have the meaning commonly understood by one of ordinary skill in the art to which this disclosure relates. That is, terms used herein have their ordinary meaning, which is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name, and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “—O-alkyl,” etc.

As used herein, and throughout this disclosure, the following terms, unless otherwise indicated, shall be understood to have the following meanings:

As used herein, including the appended claims, the singular forms of words, such as “a,” “an,” and “the,” include their corresponding plural references unless the context clearly dictates otherwise. In particular, “a,” “an,” and “the” item each include a single item selected from a list as well as mixtures of two or more items selected from the list. Thus, for example, reference to “a polypeptide” includes more than one polypeptide.

As used herein, the terms “at least one” item or “one or more” item each include a single item selected from the list as well as mixtures of two or more items selected from the list. For example, “at least one S-methyl-5-thioribose kinase polypeptide” (alternatively referred to as “S-methyl-5-thioribose kinase polypeptides”, “at least one S-methyl-5-thioribose kinase enzyme,” “S-methyl-5-thioribose kinase enzymes,” “at least one MTR kinase polypeptide”, “MTR polypeptides”, “at least one MTR kinase,” “MTR kinases,” “at least one MTR kinase enzyme,” or “MTR kinase enzymes”) refers to a single MTR kinase as well as to mixtures of two or more different MTR kinases. Similarly, the terms “at least two” items and “two or more” items each include mixtures of two items selected from the list as well as mixtures of three or more items selected from the list.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition.

Throughout this specification and claims, the word “comprise,” or variations such as “comprises” or “comprising” will be understood to imply the inclusion of a stated integer or group of integers but not the exclusion of any other integer or group of integers. The terms “comprise,” “comprises,” “comprising,” “include,” “includes,” and “including” are interchangeable and not intended to be limiting. Thus, as used herein, “comprising” and its cognates are used in their inclusive sense (i.e., equivalent to the term “including” and its corresponding cognates). Unless otherwise required by context, singular terms shall include pluralities and plural terms shall include the singular. Any example(s) following the term “e.g.” or “for example” is not meant to be exhaustive or limiting. It is understood that wherever embodiments are described herein with the language “comprising,” otherwise analogous embodiments described in terms of “consisting of” and/or “consisting essentially of” are also provided.

Unless expressly stated to the contrary, all ranges cited herein are inclusive; i.e., the range includes the values for the upper and lower limits of the range as well as all values in between. All ranges also are intended to include all included sub-ranges, although not necessarily explicitly set forth. As an example, temperature ranges, percentages, ranges of equivalents, and the like described herein include the upper and lower limits of the range and any value in the continuum there between. Numerical values provided herein, and the use of the term “about”, may include variations of ±1%, ±2%, ±3%, ±4%, ±5%, and ±10% and their numerical equivalents. The term “about” means an acceptable error for a particular value, which means, in some instances, within 0.05%, 0.5%, 1.0%, or 2.0%. In some instances, “about” means within 1, 2, 3, or 4 standard deviations of a given value. “About” when used to modify a numerically defined parameter means that the parameter may vary by as much as 10% below or above the stated numerical value for that parameter; where appropriate, the stated parameter may be rounded to the nearest whole number. For example, an amount of about 5 mg may vary between 4.5 mg and 5.5 mg. In addition, the term “or,” as used herein, denotes alternatives that may, where appropriate, be combined; that is, the term “or” includes each listed alternative separately as well as their combination.

“Protein,” “polypeptide,” and “peptide” are used interchangeably herein to denote a polymer of at least two amino acids covalently linked by an amide bond, regardless of length or post-translational modification (e.g., glycosylation or phosphorylation, lipidation, myristilation, ubiquitination, etc.)

“Amino acid” or “residue” as used in context of the polypeptides disclosed herein refers to the specific monomer at a sequence position. Amino acids are referred to herein by either their commonly known three-letter symbols or by the one-letter symbols recommended by IUPAC-IUB Biochemical Nomenclature Commission. Nucleotides, likewise, may be referred to by their commonly accepted single letter codes.

The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glutamine (Gln or Q), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V).

The abbreviations used for the genetically encoding nucleosides are conventional and are as follows: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When nucleic acid sequences are presented as a string of one-letter abbreviations, the sequences are presented in the 5′ to 3′ direction in accordance with common convention, and the phosphates are not indicated.

“Derived from” as used herein in the context of MTR kinase polypeptide, identifies the originating MTR kinase polypeptide, and/or the gene encoding such MTR kinase enzyme, upon which the MTR polypeptide was based. For example, the MTR kinase of SEQ ID NO: 7 was obtained by artificially evolving, over multiple generations the gene encoding the MTR polypeptide of SEQ ID NO: 1. Thus, this evolved MTR polypeptide is “derived from” the MTR kinase of SEQ ID NO: 1.

“Hydrophilic amino acid or residue” refers to an amino acid or residue having a side chain exhibiting a hydrophobicity of less than zero according to the normalized consensus hydrophobicity scale of Eisenberg et al., 1984, J. MOL. BIOL. 179:125-142. Genetically encoded hydrophilic amino acids include L-Thr (T), L-Ser (S), L-His (H), L-Glu (E), L-Asn (N), L-Gln (Q), L-Asp (D), L-Lys (K), and L-Arg (R).

“Acidic amino acid or residue” refers to a hydrophilic amino acid or residue having a side chain exhibiting a pK value of less than about 6 when the amino acid is included in a peptide or polypeptide. Acidic amino acids typically have negatively charged side chains at physiological pH due to loss of a hydrogen ion. Genetically encoded acidic amino acids include L-Glu (E) and L-Asp (D).

“Basic amino acid or residue” refers to a hydrophilic amino acid or residue having a side chain exhibiting a pKa value of greater than about 6 when the amino acid is included in a peptide or polypeptide. Basic amino acids typically have positively charged side chains at physiological pH due to association with hydronium ion. Genetically encoded basic amino acids include L-Arg (R) and L-Lys (K).

“Polar amino acid or residue” refers to a hydrophilic amino acid or residue having a side chain that is uncharged at physiological pH, but which has at least one bond in which the pair of electrons shared in common by two atoms is held more closely by one of the atoms. Genetically encoded polar amino acids include L-Asn (N), L-Gln (Q), L-Ser (S), and L-Thr (T).

“Non-polar amino acid or residue” refers to a hydrophobic amino acid or residue that has a side chain that is uncharged at physiological pH and that has bonds in which the pair of electrons shared in common by two atoms is generally held equally by each of the two atoms (i.e., the side chain is not polar). Genetically encoded non-polar amino acids include L-Gly (G), L-Leu (L), L-Val (V), L-Ile (I), L-Met (M), and L-Ala (A).

“Hydrophobic amino acid or residue” refers to an amino acid or residue having a side chain exhibiting a hydrophobicity of greater than zero according to the normalized consensus hydrophobicity scale of Eisenberg et al., 1984, J. MOL. BIOL. 179:125-142. Genetically encoded hydrophobic amino acids include L-Pro (P), L-Ile (I), L-Phe (F), L-Val (V), L-Leu (L), L-Trp (W), L-Met (M), L-Ala (A), and L-Tyr (Y).

“Aromatic amino acid or residue” refers to a hydrophilic or hydrophobic amino acid or residue having a side chain that includes at least one aromatic or heteroaromatic ring. Genetically encoded aromatic amino acids include L-Phe (F), L-Tyr (Y), L-His (H), and L-Trp (W). L-His (H) histidine is also classified herein as a hydrophilic residue or as a constrained residue.

As used herein, “constrained amino acid or residue” refers to an amino acid or residue that has a constrained geometry. Herein, constrained residues include L-Pro (P) and L-His (H). Histidine has a constrained geometry because it has a relatively small imidazole ring. Proline has a constrained geometry because it also has a five-membered ring.

As used herein, “aliphatic amino acid or residue” refers to a hydrophobic amino acid or residue having an aliphatic hydrocarbon side chain. Genetically encoded aliphatic amino acids include L-Ala (A), L-Val (V), L-Leu (L), and L-Ile (I).

The ability of L-Cys (C) (and other amino acids with —SH containing side chains) to exist in a peptide in either the reduced free —SH or oxidized disulfide-bridged form affects whether L-Cys (C) contributes net hydrophobic or hydrophilic character to a peptide. While L-Cys (C) exhibits a hydrophobicity of 0.29 according to the normalized consensus scale of Eisenberg (Eisenberg et al., 1984, supra), it is to be understood that for purposes of the present disclosure, L-Cys (C) is categorized into its own unique group. It is noted that cysteine (or “L-Cys” or “[C]”) is unusual in that it can form disulfide bridges with other L-Cys (C) amino acids or other sulfanyl- or sulfhydryl-containing amino acids. The “cysteine-like residues” include cysteine and other amino acids that contain sulfhydryl moieties that are available for formation of disulfide bridges.

As used herein, “small amino acid or residue” refers to an amino acid or residue having a side chain that is composed of a total three or fewer carbon and/or heteroatoms (excluding the α-carbon and hydrogens). The small amino acids or residues may be further categorized as aliphatic, non-polar, polar or acidic small amino acids or residues, in accordance with the above definitions. Genetically-encoded small amino acids include L-Ala (A), L-Val (V), L-Cys (C), L-Asn (N), L-Ser (S), L-Thr (T), and L-Asp (D).

“Hydroxyl-containing amino acid or residue” refers to an amino acid containing a hydroxyl (—OH) moiety. Genetically-encoded hydroxyl-containing amino acids include L-Ser (S) L-Thr (T), and L-Tyr (Y).

As used herein, “polynucleotide” and “nucleic acid” refer to two or more nucleotides that are covalently linked together. The polynucleotide may be wholly comprised of ribonucleotides (i.e., RNA), wholly comprised of 2′ deoxyribonucleotides (i.e., DNA), or comprised of mixtures of ribo- and 2′ deoxyribonucleotides. While the nucleosides will typically be linked together via standard phosphodiester linkages, the polynucleotides may include one or more non-standard linkages. The polynucleotide may be single-stranded or double-stranded, or the polynucleotide may include both single-stranded regions and double-stranded regions. Moreover, while a polynucleotide will typically be composed of the naturally occurring encoding nucleobases (i.e., adenine, guanine, uracil, thymine and cytosine), it may include one or more modified and/or synthetic nucleobases, such as, for example, inosine, xanthine, hypoxanthine, etc. In some embodiments, such modified or synthetic nucleobases are nucleobases encoding amino acid sequences.

As used herein, “nucleoside” refers to glycosylamines comprising a nucleobase (i.e., a nitrogenous base), and a 5-carbon sugar (e.g., ribose or deoxyribose). Non-limiting examples of nucleosides include cytidine, uridine, adenosine, guanosine, thymidine, and inosine. In contrast, the term “nucleotide” refers to the glycosylamines comprising a nucleobase, a 5-carbon sugar, and one or more phosphate groups. In some embodiments, nucleosides can be phosphorylated by kinases to produce nucleotides.

As used herein, “nucleoside diphosphate” refers to glycosylamines comprising a nucleobase (i.e., a nitrogenous base), a 5-carbon sugar (e.g., ribose or deoxyribose), and a diphosphate (i.e., pyrophosphate) moiety. In some embodiments herein, “nucleoside diphosphate” is abbreviated as “NDP.” Non-limiting examples of nucleoside diphosphates include cytidine diphosphate (CDP), uridine diphosphate (UDP), adenosine diphosphate (ADP), guanosine diphosphate (GDP), thymidine diphosphate (TDP), and inosine diphosphate (IDP). The terms “nucleoside” and “nucleotide” may be used interchangeably in some contexts.

As used herein, “nucleoside triphosphate” refers to glycosylamines comprising a nucleobase (i.e., a nitrogenous base), a 5-carbon sugar (e.g., ribose or deoxyribose), and a triphosphate moiety. In some embodiments herein, “nucleoside triphosphate” is abbreviated as “NTP.” Non-limiting examples of nucleoside triphosphates include cytidine triphosphate (CTP), uridine triphosphate (UTP), adenosine triphosphate (ATP), guanosine triphosphate (GTP), thymidine triphosphate (TTP), and inosine triphosphate (ITP). The terms “nucleoside” and “nucleotide” may be used interchangeably in some contexts.

As used herein, “conservative amino acid substitution” refers to a substitution of a residue with a different residue having a similar side chain, and thus typically involves substitution of the amino acid in the polypeptide with amino acids within the same or similar defined class of amino acids. By way of example and not limitation, in some embodiments, an amino acid with an aliphatic side chain is substituted with another aliphatic amino acid (e.g., alanine, valine, leucine, and isoleucine); an amino acid with an hydroxyl side chain is substituted with another amino acid with an hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid and glutamic acid); and/or a hydrophobic or hydrophilic amino acid is replaced with another hydrophobic or hydrophilic amino acid, respectively.

As used herein, “non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

As used herein, “deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an evolved enzyme. Deletions can be directed to the internal portions and/or terminal portions of the polypeptide. In various embodiments, the deletion can comprise a continuous segment or can be discontinuous. Deletions are typically indicated by “-” in amino acid sequences.

As used herein, “insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

The term “amino acid substitution set” or “substitution set” refers to a group of amino acid substitutions in a polypeptide sequence, as compared to a reference sequence. A substitution set can have 1, 2, 3, 4, 5, 6, 7, 8, 9, 10, 11, 12, 13, 14, 15, or more amino acid substitutions.

A “functional fragment” and “biologically active fragment” are used interchangeably herein to refer to a polypeptide that has an amino-terminal and/or carboxy-terminal deletion(s) and/or internal deletions, but where the remaining amino acid sequence is identical to the corresponding positions in the sequence to which it is being compared and that retains substantially all of the activity of the full-length polypeptide.

As used herein, “isolated polypeptide” refers to a polypeptide which is substantially separated from other contaminants that naturally accompany it (e.g., protein, lipids, and polynucleotides). The term embraces polypeptides which have been removed or purified from their naturally occurring environment or expression system (e.g., within a host cell or via in vitro synthesis). The recombinant MTR kinase polypeptides may be present within a cell, present in the cellular medium, or prepared in various forms, such as lysates or isolated preparations. As such, in some embodiments, the recombinant MTR kinase polypeptides can be an isolated polypeptide.

As used herein, “substantially pure polypeptide” or “purified protein” refers to a composition in which the polypeptide species is the predominant species present (i.e., on a molar or weight basis it is more abundant than any other individual macromolecular species in the composition) and is generally a substantially purified composition when the object species comprises at least about 50 percent of the macromolecular species present by mole or % weight. However, in some embodiments, the composition comprising MTR kinase comprises MTR kinase that is less than 50% pure (e.g., about 10%, about 20%, about 30%, about 40%, or about 50%). Generally, a substantially pure MTR kinase composition comprises about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated recombinant MTR kinase polypeptides are substantially pure polypeptide compositions.

“Improved enzyme property” refers to an MTR kinase that exhibits an improvement in any enzyme property as compared to a reference MTR kinase. For the MTR kinases described herein, the comparison is generally made to the wild-type MTR kinase polypeptide, although in some embodiments, the reference MTR kinase can be another improved MTR kinase. Enzyme properties for which improvement is desirable include, but are not limited to, enzymatic activity (which can be expressed in terms of percent conversion of the substrate), thermal stability, pH activity profile, cofactor requirements, refractoriness to inhibitors (e.g., product inhibition), stereospecificity, and stereoselectivity (including enantioselectivity).

“Increased enzymatic activity” refers to an improved property of the MTR kinases, which can be represented by an increase in specific activity (e.g., product produced/time/weight protein) or an increase in percent conversion of the substrate to the product (e.g., percent conversion of starting amount of substrate to product in a specified time period using a specified amount of MTR kinase) as compared to the reference MTR kinase. Exemplary methods to determine enzyme activity are provided in the Examples. Any property relating to enzyme activity may be affected, including the classical enzyme properties of Kₘ, Vₘₐₓ or k_{cat}, changes of which can lead to increased enzymatic activity. Improvements in enzyme activity can be from about 1.5 times the enzymatic activity of the corresponding wild-type MTR kinase polypeptide, to as much as 2 times, 5 times, 10 times, 20 times, 25 times, 50 times, 75 times, 100 times, 150 times, 200 times, 500 times, 1000 times, 3000 times, 5000 times, 7000 times or more enzymatic activity than the naturally occurring MTR kinase or another MTR kinase from which the MTR kinase polypeptides were derived. In specific embodiments, the MTR kinase polypeptide exhibits improved enzymatic activity in the range of 150 to 3000 times, 3000 to 7000 times, or more than 7000 times greater than that of the parent MTR kinase polypeptide. It is understood by the skilled artisan that the activity of any enzyme is diffusion limited such that the catalytic turnover rate cannot exceed the diffusion rate of the substrate, including any required cofactors. The theoretical maximum of the diffusion limit, or k_{ca}/Kₘ, is generally about 10⁸ to 10⁹ (M⁻¹s⁻¹). Hence, any improvements in the enzyme activity of the MTR kinase will have an upper limit related to the diffusion rate of the substrates acted on by the MTR kinase enzyme. MTR kinase activity can be measured by any one of standard assays used for measuring kinase activity, or via a coupled assay with a nucleoside phosphorylase enzyme that is capable of catalyzing reaction between the MTR kinase product and a nucleoside base to afford a nucleoside, or by any of the traditional methods for assaying chemical reactions, including but not limited to HPLC, HPLC-MS, UPLC, UPLC-MS, TLC, and NMR. Comparisons of enzyme activities are made using a defined preparation of enzyme, a defined assay under a set condition, and one or more defined substrates, as further described in detail herein. Generally, when lysates are compared, the numbers of cells and the amount of protein assayed are determined as well as use of identical expression systems and identical host cells to minimize variations in amount of enzyme produced by the host cells and present in the lysates.

As used herein, a “vector” is a DNA construct for introducing a DNA sequence into a cell. In some embodiments, the vector is an expression vector that is operably linked to a suitable control sequence capable of effecting the expression in a suitable host of the polypeptide encoded in the DNA sequence. In some embodiments, an “expression vector” has a promoter sequence operably linked to the DNA sequence (e.g., transgene) to drive expression in a host cell, and in some embodiments, also comprises a transcription terminator sequence.

As used herein, the term “expression” includes any step involved in the production of the polypeptide including, but not limited to, transcription, post-transcriptional modification, translation, and post-translational modification. In some embodiments, the term also encompasses secretion of the polypeptide from a cell.

As used herein, the term “produces” refers to the production of proteins and/or other compounds by cells. It is intended that the term encompasses any step involved in the production of polypeptides including, but not limited to, transcription, post-transcriptional modification, translation, and post-translational modification. In some embodiments, the term also encompasses secretion of the polypeptide from a cell.

As used herein, an amino acid or nucleotide sequence (e.g., a promoter sequence, signal peptide, terminator sequence, etc.) is “heterologous” to another sequence with which it is operably linked if the two sequences are not associated in nature. For example, a “heterologous polynucleotide” is any polynucleotide that is introduced into a host cell by laboratory techniques, and the term includes polynucleotides that are removed from a host cell, subjected to laboratory manipulation, and then reintroduced into a host cell.

As used herein, the terms “host cell” and “host strain” refer to suitable hosts for expression vectors comprising DNA provided herein (e.g., the polynucleotides encoding the MTR kinase variants). In some embodiments, the host cells are prokaryotic or eukaryotic cells that have been transformed or transfected with vectors constructed using recombinant DNA techniques as known in the art.

The term “analogue” means a polypeptide having more than 70% sequence identity but less than 100% sequence identity (e.g., more than 75%, 78%, 80%, 83%, 85%, 88%, 90%, 91%, 92%, 93%, 94%, 95%, 96%, 97%, 98%, or 99% sequence identity) with a reference polypeptide. In some embodiments, “analogues” means polypeptides that contain one or more non-naturally occurring amino acid residues including, but not limited, to homoarginine, ornithine and norvaline, as well as naturally occurring amino acids. In some embodiments, analogues also include one or more D-amino acid residues and non-peptide linkages between two or more amino acid residues.

As used herein, “EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

As used herein, “ATCC” refers to the American Type Culture Collection whose biorepository collection includes genes and strains.

As used herein, “NCBI” refers to National Center for Biological Information and the sequence databases provided therein.

“Coding sequence” refers to that portion of a nucleic acid (e.g., a gene) that encodes an amino acid sequence of a protein.

“Naturally occurring” or “wild-type” refers to a form found in nature. For example, a naturally occurring or wild-type polypeptide or polynucleotide sequence is a sequence present in an organism that can be isolated from a source in nature and that has not been intentionally modified by human manipulation. Herein, “wild-type” polypeptide or polynucleotide sequences may be denoted “WT”.

“Recombinant” when used with reference to, e.g., a cell, nucleic acid, or polypeptide, refers to a material, or a material corresponding to the natural or native form of the material, that has been modified in a manner that would not otherwise exist in nature, or is identical thereto but produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Percentage of sequence identity,” “percent identity,” and “percent identical” are used herein to refer to comparisons between polynucleotide sequences or polypeptide sequences and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Determination of optimal alignment and percent sequence identity is performed using the BLAST and BLAST 2.0 algorithms (see e.g., Altschul et al., 1990, J. MOL. BIOL. 215: 403-410; and Altschul et al., 1977, NUCLEIC ACIDS RES. 3389-3402). Software for performing BLAST analyses is publicly available through the National Center for Biotechnology

Information website.

Briefly, the BLAST analyses involve first identifying high scoring sequence pairs (HSPs) by identifying in the query sequence short words of length W, which either match or satisfy some positive-valued threshold score T when aligned with a word of the same length in a database sequence. T is referred to as, the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff and Henikoff, 1989, PROC. NATL. ACAD. Sci. USA 89:10915).

Numerous other algorithms are available that function similarly to BLAST in providing percent identity for two sequences. Optimal alignment of sequences for comparison can be conducted, e.g., by the local homology algorithm of Smith and Waterman, 1981, ADV. APPL. MATH. 2:482, by the homology alignment algorithm of Needleman and Wunsch, 1970, J. MOL. BIOL. 48:443, by the search for similarity method of Pearson and Lipman, 1988, N USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Additionally, determination of sequence alignment and percent sequence identity can employ the BESTFIT or GAP programs in the GCG Wisconsin Software package (Accelrys, Madison WI), using default parameters provided.

“Substantial identity” refers to a polynucleotide or polypeptide sequence that has at least 80 percent sequence identity, preferably at least 85 percent sequence identity, more preferably at least 89 percent sequence identity, more preferably at least 95 percent sequence identity, and even more preferably at least 99 percent sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. In specific embodiments applied to polypeptides, the term “substantial identity” means that two polypeptide sequences, when optimally aligned, such as by the programs GAP or BESTFIT using default gap weights, share at least 80 percent sequence identity, preferably at least 89 percent sequence identity, more preferably at least 95 percent sequence identity or more (e.g., 99 percent sequence identity). Preferably, residue positions which are not identical differ by conservative amino acid substitutions.

“Corresponding to”, “reference to” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an MTR kinase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Stereoselectivity” refers to the preferential formation in a chemical or enzymatic reaction of one stereoisomer over another. Stereoselectivity can be partial, where the formation of one stereoisomer is favored over the other, or it may be complete where only one stereoisomer is formed. When the stereoisomers are enantiomers, the stereoselectivity is referred to as enantioselectivity, the fraction (typically reported as a percentage) of one enantiomer in the sum of both. It is commonly alternatively reported in the art (typically as a percentage) as the enantiomeric excess (EE) calculated therefrom according to the formula (major enantiomer−minor enantiomer)/(major enantiomer+minor enantiomer). Where the stereoisomers are diastereoisomers, the stereoselectivity is referred to as diastereoselectivity, the fraction (typically reported as a percentage) of one diastereomer in a mixture of two diastereomers, commonly alternatively reported as the diastereomeric excess (DE). Enantiomeric excess and diastereomeric excess are types of stereomeric excess.

“Highly stereoselective” refers to a chemical or enzymatic reaction that is capable of converting a substrate to its corresponding product with at least about 85% stereoisomeric excess.

“Chemoselectivity” refers to the preferential formation in a chemical or enzymatic reaction of one product over another.

“Conversion” refers to the enzymatic transformation of a substrate to the corresponding product. “Percent conversion” refers to the percent of the substrate that is converted to the product within a period of time under specified conditions. Thus, for example, the “enzymatic activity” or “activity” of an MTR kinase polypeptide can be expressed as “percent conversion” of the substrate to the product.

“Chiral alcohol” refers to amines of general formula R¹—CH(OH)—R² wherein R¹ and R² are nonidentical and is employed herein in its broadest sense, including a wide variety of aliphatic and alicyclic compounds of different, and mixed, functional types, characterized by the presence of a primary hydroxyl group bound to a secondary carbon atom which, in addition to a hydrogen atom, carries either (i) a divalent group forming a chiral cyclic structure, or (ii) two substituents (other than hydrogen) differing from each other in structure or chirality. Divalent groups forming a chiral cyclic structure include, for example, 2-methylbutane-1,4-diyl, pentane-1,4-diyl, hexane-1,4-diyl, hexane-1,5-diyl, 2-methylpentane-1,5-diyl. The two different substituents on the secondary carbon atom (R¹ and R² above) also can vary widely and include alkyl, aralkyl, aryl, halo, hydroxy, lower alkyl, lower alkoxy, lower alkylthio, cycloalkyl, carboxy, carboalkoxy, carbamoyl, mono- and di-(lower alkyl) substituted carbamoyl, trifluoromethyl, phenyl, nitro, amino, mono- and di-(lower alkyl) substituted amino, alkylsulfonyl, arylsulfonyl, alkylcarboxamido, arylcarboxamido, etc., as well as alkyl, aralkyl, or aryl substituted by the foregoing.

Immobilized enzyme preparations have a number of recognized advantages. They can confer shelf life to enzyme preparations, they can improve reaction stability, they can enable stability in organic solvents, they can aid in protein removal from reaction streams, as examples. “Stable” refers to the ability of the immobilized enzymes to retain their structural conformation and/or their activity in a solvent system that contains organic solvents. Stable immobilized enzymes lose less than 10% activity per hour in a solvent system that contains organic solvents. Stable immobilized enzymes lose less than 9% activity per hour in a solvent system that contains organic solvents. Preferably, the stable immobilized enzymes lose less than 8% activity per hour in a solvent system that contains organic solvents. Preferably, the stable immobilized enzymes lose less than 7% activity per hour in a solvent system that contains organic solvents. Preferably, the stable immobilized enzymes lose less than 6% activity per hour in a solvent system that contains organic solvents. Preferably, the stable immobilized enzymes lose less than 5% activity per hour in a solvent system that contains organic solvents. Preferably, the stable immobilized enzymes less than 4% activity per hour in a solvent system that contains organic solvents. Preferably, the stable immobilized enzymes lose less than 3% activity per hour in a solvent system that contains organic solvents. Preferably, the stable immobilized enzymes lose less than 2% activity per hour in a solvent system that contains organic solvents. Preferably, the stable immobilized enzymes lose less than 1% activity per hour in a solvent system that contains organic solvents.

“Thermostable” refers to an MTR kinase polypeptide that maintains similar activity (more than 60% to 80% for example) after exposure to elevated temperatures (e.g., 40-80° C.) for a period of time (e.g. 0.5-24 hours) compared to the untreated enzyme.

“Solvent stable” refers to an MTR kinase polypeptide that maintains similar activity (more than e.g., 60% to 80%) after exposure to varying concentrations (e.g., 5-99%) of solvent (isopropyl alcohol, tetrahydrofuran, 2-methyltetrahydrofuran, acetone, toluene, butylacetate, methyl tert-butylether, etc.) for a period of time (e.g., 0.5-24 hours) compared to the untreated enzyme.

“pH stable” refers to an MTR kinase polypeptide that maintains similar activity (more than e.g., 60% to 80%) after exposure to high or low pH (e.g., 4.5-6 or 8 to 12) for a period of time (e.g., 0.5-24 hours) compared to the untreated enzyme.

“Thermo- and solvent stable” refers to an MTR kinase polypeptide that is both thermostable and solvent stable.

As used herein, the terms “biocatalysis,” “biocatalytic,” “biotransformation,” and “biosynthesis” refer to the use of enzymes to perform chemical reactions on organic compounds.

The term “effective amount” means an amount sufficient to produce the desired result. One of general skill in the art may determine what the effective amount by using routine experimentation.

The terms “isolated” and “purified” are used to refer to a molecule (e.g., an isolated nucleic acid, polypeptide, etc.) or other component that is removed from at least one other component with which it is naturally associated. The term “purified” does not require absolute purity, rather it is intended as a relative definition.

“S-methylthioribose kinase” or “MTR kinase”, as used herein, refers to a polypeptide having an enzymatic capability of phosphorylating of S-methyl-5-thioribose. The polypeptide may use a cofactor, such as a divalent cation, including Mg²⁺, Ca²⁺, Mn²⁺, or Co²⁺, and uses ATP or another nucleotide triphosphate as a cofactor from which it transfers a phosphate reside to the substrate. MTR kinases, as used herein, include naturally occurring (wild-type) MTR kinases as well as non-naturally occurring polypeptides generated by human manipulation.

Those skilled in the art will recognize that chiral compounds, and in particular sugars, can be drawn in a number of different ways that are equivalent. Those skilled in the art will further recognize that the identity and regiochemical position of the substituents on ribose can vary widely and that the same principles of stereochemical equivalence apply regardless of substituent. Non-limiting examples of such equivalence include those exemplified below.

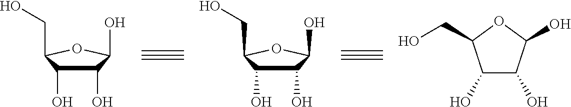

### MTR Kinases

This disclosure relates to MTR kinase polypeptides capable of phosphorylating ribose and ribose derivatives to the corresponding alpha-1-phosphate sugar (or a salt thereof), which is a substrate for another nucleoside phosphorylase enzyme that can then be used to convert the 1-phosphate to a nucleoside in the synthesis of nucleosides, particularly 5-isobutyrylribose, which can be converted to 5′-isobutyryl nucleosides, particularly 5′-isobutyryluridine. In embodiments, the MTR kinases are capable of the following conversion:

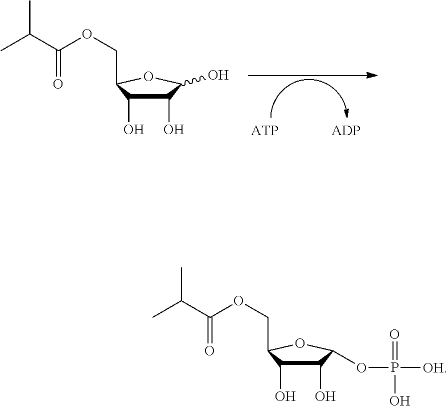

In particular embodiments, the MTR kinase polypeptides are capable of the following conversion:

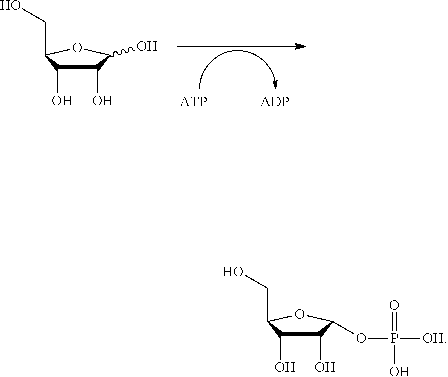

In such embodiments, the phosphorylated compound may be in the form of a salt.

In embodiments, the MTR kinases described herein preferentially phosphorylate ribose and 5-isobutyrylribose to the alpha form. The alpha form of D-ribose 1-phosphate is shown here for illustrative purposes.

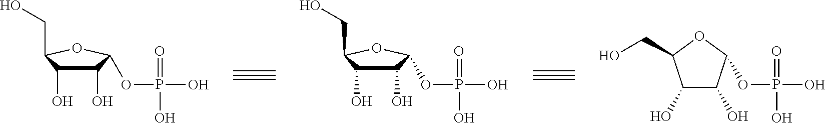

The beta form of D-ribose 1-phosphate is also shown here for comparison and for illustrative purposes.

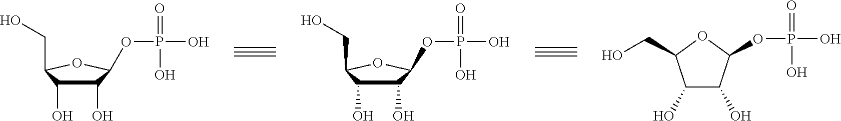

In embodiments, the MTR kinase polypeptides described herein have an amino acid sequence that has one or more amino acid differences as compared to a reference amino acid sequence of a wild-type MTR kinase or a modified MTR kinase that result in an improved property of the enzyme for the defined substrate.

The MTR kinase polypeptides described herein are the product of directed evolution from a commercially available, wild-type MTR kinase, which has been characterized as an MTR kinase (see Kenneth A. Cornell et al., 317 BIOCHEM. J. 285-290 (1996)) and which has the amino acid sequence as set forth below in SEQ ID NO: 1.

(SEQ ID NO: 1)
MSQYHTFTAHDAVAYAQQFAGIDNPSELVSAQEVGDGNLNLVFKVFDRQ
GVSRAIVKQALPYVRCVGESWPLTLDRARLEAQTLVAHYQHSPQHTVKI
HHFDPELAVMVMEDLSDHRIWRGELIANVYYPQAARQLGDYLAQVLFHT
SDFYLHPHEKKAQVAQFINPAMCEITEDLFFNDPYQIHERNNYPAELEA
DVAALRDDAQLKLAVAALKHRFFAHAEALLHGDIHSGSIFVAEGSLKAI
DAEFGYFGPIGFDIGTAIGNLLLNYCGLPGQLGIRDAAAAREQRLNDIH
QLWTTFAERFQALAAEKTRDAALAYPGYASAFLKKVWADAVGFCGSELI
RRSVGLSHVADIDTIQDDAMRHECLRHAITLGRALIVLAERIDSVDELL
ARVRQYS

In specific occurrences, the wild-type MTR kinase may be encoded by the DNA sequence as set forth below in SEQ ID NO: 2.

(SEQ ID NO: 2)
ATGAGCCAGTATCATACCTTCACCGCGCATGATGCGGTGGCGTATGCGC
AGCAATTTGCGGGCATTGATAACCCGAGCGAGCTGGTTAGCGCGCAAGA
AGTTGGTGACGGCAACCTGAACCTGGTGTTCAAGGTTTTTGATCGTCAG
GGTGTGAGCCGTGCGATCGTTAAACAAGCGCTGCCGTACGTGCGTTGCG
TTGGTGAAAGCTGGCCGCTGACCCTGGACCGTGCGCGTCTGGAAGCGCA
GACCCTGGTGGCGCACTATCAGCACAGCCCGCAACACACCGTTAAGATC
CACCACTTCGATCCGGAGCTGGCGGTGATGGTTATGGAAGACCTGAGCG
ATCACCGTATTTGGCGTGGTGAGCTGATCGCGAACGTGTACTATCCGCA
GGCGGCGCGTCAACTGGGTGACTACCTGGCGCAGGTTCTGTTCCACACC
AGCGATTTTTATCTGCACCCGCACGAGAAGAAAGCGCAGGTGGCGCAAT
TCATTAACCCGGCGATGTGCGAGATCACCGAAGACCTGTTCTTTAACGA
TCCGTACCAGATTCACGAACGTAACAACTATCCGGCGGAGCTGGAAGCG
GATGTGGCGGCGCTGCGTGATGATGCGCAACTGAAGCTGGCGGTTGCGG
CGCTGAAACACCGTTTCTTTGCGCATGCGGAGGCGCTGCTGCATGGTGA
CATTCACAGCGGCAGCATCTTCGTTGCGGAGGGTAGCCTGAAGGCGATC
GACGCGGAATTCGGTTACTTTGGCCCGATCGGTTTTGATATTGGTACCG
CGATCGGCAACCTGCTGCTGAACTATTGCGGTCTGCCGGGTCAACTGGG
TATTCGTGATGCGGCGGCGGCGCGTGAACAGCGTCTGAACGATATCCAC
CAACTGTGGACCACCTTCGCGGAGCGTTTTCAAGCGCTGGCGGCGGAAA
AGACCCGTGACGCGGCGCTGGCGTACCCGGGTTATGCGAGCGCGTTCCT
GAAGAAAGTGTGGGCGGATGCGGTTGGTTTTTGCGGCAGCGAGCTGATT
CGTCGTAGCGTGGGCCTGAGCCACGTTGCGGACATCGATACCATTCAGG
ACGATGCGATGCGTCACGAATGCCTGCGTCACGCGATCACCCTGGGTCG
TGCGCTGATTGTTCTGGCGGAGCGTATCGACAGCGTGGATGAACTGCTG
GCGCGTGTTCGTCAATACAGC

In embodiments, the MTR kinase polypeptides of the disclosure may demonstrate improvements relative the MTR kinases of SEQ ID NO: 1, such as increases in enzyme activity, stereoselectivity, stereospecificity, thermostability, solvent stability, or phosphorylated product inhibition.

In embodiments, the MTR kinase polypeptides of the disclosure may demonstrate improvements in the rate of enzymatic activity, i.e., the rate of converting the substrate to the product. In some embodiments, the MTR kinases are polypeptides are capable of converting the substrate to the product at a rate that is at least 1.5-times, 2-times, 3-times, 4-times, 5-times, 10-times, 25-times, or 50-times the rate exhibited by the enzyme of SEQ ID NO: 1.

In some embodiments, such MTR kinases are polypeptides that are also capable of converting the substrate to the product with a percent diastereometric excess of at least about 80%. In some embodiments, such MTR kinases are polypeptides that are also capable of converting the substrate to the product with a percent diastereometric excess of at least about 90%. In some embodiments, such MTR kinases are polypeptides that are also capable of converting the substrate to the product with a percent diastereometric excess of at least about 99%.

In some embodiments, the MTR kinase polypeptide is highly stereoselective, wherein the polypeptide can phosphorylate the substrate to the product in greater than about 99%, 99.1%, 99.2%, 99.3%, 99.4%, 99.5%, 99.6%, 99.7%, 99.8%, or 99.9% diastereometric excess.

In embodiments, the MTR kinases described herein include polypeptides having the amino acid sequence as set forth below in SEQ ID NO: 3.

(SEQ ID NO: 3)
MSQYHTFTAHDAVAYAQQFAGIDNPSELVSAQEVGDGNLNLVFKVFDRQ
GVSRAIVKQALPYVRCVGESWPLTLDRARHEAQTLVAHYQHSPQHTVKI
HHFDPELAVMVMEDLSDHRIWRGELIANVYYPQAARQLGDYLAQVLFHT
SDFYLHPHEKKAQVAQFINPAMCEITEDLFFNDPYQIHERNNYPAELEA
DVAALRDDAQLKLAVAALKHRFFAHAEALLHGDIHSGSIFVAEGSLKAI
DAEFGYFGPIGFDIGTAIGNLLLNYCGLPGQLGIRDAAAAREQRLNDIH
QLWTTFAERFQALAAEKTRDAALAYPGYASAFLKKVWADAVGFCGSELI
RRSVGLSHVADIDTIQDDAMRHECLRHAITLGRALIVLAERIDSVDELL
ARVRQYSLEHHHHHH

In specific occurrences, the polypeptides comprising the amino acid sequence as set forth above in SEQ ID NO: 3 may be encoded by the DNA sequence as set forth below in SEQ ID NO: 4.

(SEQ ID NO: 4)
ATGAGCCAGTATCATACCTTCACCGCGCATGATGCGGTGGCGTATGCGC
AGCAATTTGCGGGCATTGATAACCCGAGCGAGCTGGTTAGCGCGCAAGA
AGTTGGTGACGGCAACCTGAACCTGGTGTTCAAGGTTTTTGATCGTCAG
GGTGTGAGCCGTGCGATCGTTAAACAAGCGCTGCCGTACGTGCGTTGCG
TTGGTGAAAGCTGGCCGCTGACCCTGGACCGTGCGCGTCATGAAGCGCA
GACCCTGGTGGCGCACTATCAGCACAGCCCGCAACACACCGTTAAGATC
CACCACTTCGATCCGGAGCTGGCGGTGATGGTTATGGAAGACCTGAGCG
ATCACCGTATTTGGCGTGGTGAGCTGATCGCGAACGTGTACTATCCGCA
GGCGGCGCGTCAACTGGGTGACTACCTGGCGCAGGTTCTGTTCCACACC
AGCGATTTTTATCTGCACCCGCACGAGAAGAAAGCGCAGGTGGCGCAAT
TCATTAACCCGGCGATGTGCGAGATCACCGAAGACCTGTTCTTTAACGA
TCCGTACCAGATTCACGAACGTAACAACTATCCGGCGGAGCTGGAAGCG
GATGTGGCGGCGCTGCGTGATGATGCGCAACTGAAGCTGGCGGTTGCGG
CGCTGAAACACCGTTTCTTTGCGCATGCGGAGGCGCTGCTGCATGGTGA
CATTCACAGCGGCAGCATCTTCGTTGCGGAGGGTAGCCTGAAGGCGATC
GACGCGGAATTCGGTTACTTTGGCCCGATCGGTTTTGATATTGGTACCG
CGATCGGCAACCTGCTGCTGAACTATTGCGGTCTGCCGGGTCAACTGGG
TATTCGTGATGCGGCGGCGGCGCGTGAACAGCGTCTGAACGATATCCAC
CAACTGTGGACCACCTTCGCGGAGCGTTTTCAAGCGCTGGCGGCGGAAA
AGACCCGTGACGCGGCGCTGGCGTACCCGGGTTATGCGAGCGCGTTCCT
GAAGAAAGTGTGGGCGGATGCGGTTGGTTTTTGCGGCAGCGAGCTGATT
CGTCGTAGCGTGGGCCTGAGCCACGTTGCGGACATCGATACCATTCAGG
ACGATGCGATGCGTCACGAATGCCTGCGTCACGCGATCACCCTGGGTCG
TGCGCTGATTGTTCTGGCGGAGCGTATCGACAGCGTGGATGAACTGCTG
GCGCGTGTTCGTCAATACAGCCTCGAGCACCACCACCACCACCACTGA

In additional embodiments, the MTR kinases described herein include polypeptides having the amino acid sequence as set forth below in SEQ ID NO: 5.

(SEQ ID NO: 5)
MSQYHTFTAHDAVAYAQQFAGIDNPSELVSAQEVGDGNLNLVFKVFDRQ
GVSRAIVKQALPYPRCVGESWPLTLDRARHEAQTLVAHYQHSPQHTVKI
MHHFDPELAVVMEDLSDHRIWRGELIANVYYPQAARQLGDYLAQVLFHT
SDFYLHPHEKKAQVAQFINPAMCEISEDLIFNDPYQIHERNNYPAELEA
DVAALRDDAQLKLAVAALKHRFFAHAEALLHGDLHSGSIFVAEGSLKAI
DAEFGYFGPIGFDIGTAIGNLLLNYCGLPGQLGIRDAAAAREQRLNDIH
QLWTTFAERFQALAAEKTRDAALAYPGYASAFLKKVWADAVGFCGSELI
RRSVGLSHVADIDTIQDDAMRHECLRHAITLGRALIVLAERIDSVDELL
ARVRQYSLEHHHHHH

In specific occurrences, the polypeptides comprising the amino acid sequence as set forth above in SEQ ID NO: 5 may be encoded by the DNA sequence as set forth below in SEQ ID NO: 6.

(SEQ ID NO: 6)
ATGAGCCAGTATCATACCTTCACCGCGCATGATGCGGTGGCGTATGCGC
AGCAATTTGCGGGCATTGATAACCCGAGCGAGCTGGTTAGCGCGCAAGA
AGTTGGTGACGGCAACCTGAACCTGGTGTTCAAGGTTTTTGATCGTCAG
GGTGTGAGCCGTGCGATCGTTAAACAAGCGCTGCCGTACCCCCGTTGCG
TTGGTGAAAGCTGGCCGCTGACCCTGGACCGTGCGCGTCATGAAGCGCA
GACCCTGGTGGCGCACTATCAGCACAGCCCGCAACACACCGTTAAGATC
CACCACTTCGATCCGGAGCTGGCGGTGATGGTTATGGAAGACCTGAGCG
ATCACCGTATTTGGCGTGGTGAGCTGATCGCGAACGTGTACTATCCGCA
GGCGGCGCGTCAACTGGGTGACTACCTGGCGCAGGTTCTGTTCCACACC
AGCGATTTTTATCTGCACCCGCACGAGAAGAAAGCGCAGGTGGCGCAAT
TCATTAACCCGGCGATGTGCGAGATCAGCGAAGACCTGATTTTTAACGA
TCCGTACCAGATTCACGAACGTAACAACTATCCGGCGGAGCTGGAAGCG
GATGTGGCGGCGCTGCGTGATGATGCGCAACTGAAGCTGGCGGTTGCGG
CGCTGAAACACCGTTTCTTTGCGCATGCGGAGGCGCTGCTGCATGGTGA
CCTTCACAGCGGCAGCATCTTCGTTGCGGAGGGTAGCCTGAAGGCGATC
GACGCGGAATTCGGTTACTTTGGCCCGATCGGTTTTGATATTGGTACCG
CGATCGGCAACCTGCTGCTGAACTATTGCGGTCTGCCGGGTCAACTGGG
TATTCGTGATGCGGCGGCGGCGCGTGAACAGCGTCTGAACGATATCCAC
CAACTGTGGACCACCTTCGCGGAGCGTTTTCAAGCGCTGGCGGCGGAAA
AGACCCGTGACGCGGCGCTGGCGTACCCGGGTTATGCGAGCGCGTTCCT
GAAGAAAGTGTGGGCGGATGCGGTTGGTTTTTGCGGCAGCGAGCTGATT
CGTCGTAGCGTGGGCCTGAGCCACGTTGCGGACATCGATACCATTCAGG
ACGATGCGATGCGTCACGAATGCCTGCGTCACGCGATCACCCTGGGTCG
TGCGCTGATTGTTCTGGCGGAGCGTATCGACAGCGTGGATGAACTGCTG
GCGCGTGTTCGTCAATACAGCCTCGAGCACCACCACCACCACCACTGA

In additional embodiments, the MTR kinases described herein include polypeptides having the amino acid sequence as set forth below in SEQ ID NO: 7.

(SEQ ID NO: 7)
MSQYHTFTADDAVAYAQQFAGIDNPSELVSAQEVGDGNLNLVFKVFDRQ
GVSRAIVKQALPYPRRVGPSWPLTLDRARHEAQTLVAHYQHSPQHTVKI
HHFDPELAVMVMEDLSDHRIWRGELIANVYYPQAARQLGDYLAQVLFHT
SDFYLHPHEKKAQVAQFINPAMCEISEDLVENDPYQIHERNNYPAELEA
DVAALRDDAQLKLAVAALKHRFFAHAEALLHGDLHTGSIFVAEGSLKVI
DAEFGYFGPIGFDIGTAIGNLLLNYCGLPGQLGIRDAAAAREQRLNDIH
QLWTTFAERFQALAAEKTRDAALAYPGYASAFLKKVWADAVGFCGSELI
RRSVGLAHVADIDTIQDDAMRHECLREAITLGRALIVLAERIDSVDELL
ARVRQYSLEHHHHHH

In specific occurrences, the polypeptides comprising the amino acid sequence as set forth above in SEQ ID NO: 7 may be encoded by the DNA sequence as set forth below in SEQ ID NO: 8.

(SEQ ID NO: 8)
ATGAGCCAGTATCATACCTTCACCGCGGATGATGCGGTGGCGTATGCGC
AGCAATTTGCGGGCATTGATAACCCGAGCGAGCTGGTTAGCGCGCAAGA
AGTTGGTGACGGCAACCTGAACCTGGTGTTCAAGGTTTTTGATCGTCAG
GGTGTGAGCCGTGCGATCGTTAAACAAGCGCTGCCGTACCCCCGTCGCG
TTGGTCCGAGCTGGCCGCTGACCCTGGACCGTGCCCGTCATGAAGCGCA
GACCCTGGTTGCGCACTATCAGCACAGCCCGCAACACACCGTTAAGATC
CACCACTTCGATCCGGAGCTGGCGGTGATGGTTATGGAAGACCTGAGCG
ATCACCGTATTTGGCGTGGTGAGCTGATCGCGAACGTGTACTATCCGCA
GGCGGCGCGTCAACTGGGTGACTACCTGGCGCAGGTTCTGTTCCACACC
AGCGATTTTTATCTGCACCCGCACGAGAAGAAAGCGCAGGTGGCGCAAT
TCATTAACCCGGCCATGTGCGAAATCAGCGAAGACCTGGTGTTTAACGA
TCCGTACCAGATTCACGAACGTAACAACTATCCGGCGGAGCTGGAAGCG
GATGTGGCGGCGCTGCGTGATGATGCGCAACTGAAGCTGGCGGTTGCGG
CGCTGAAACACCGTTTCTTTGCGCATGCGGAGGCGCTGCTGCATGGTGA
CCTTCACACCGGCAGCATCTTCGTTGCGGAGGGTAGCCTGAAGGTGATC
GACGCGGAATTCGGTTACTTTGGCCCGATCGGTTTTGATATTGGTACCG
CGATCGGCAACCTGCTGCTGAACTATTGCGGTCTGCCGGGTCAACTGGG
TATTCGTGATGCGGCGGCGGCGCGTGAACAGCGTCTGAACGATATCCAC
CAACTGTGGACCACCTTCGCGGAGCGTTTTCAAGCGCTGGCGGCGGAAA
AGACCCGTGACGCGGCGCTGGCGTACCCGGGTTATGCGAGCGCGTTCCT
GAAGAAAGTGTGGGCGGATGCGGTTGGTTTTTGCGGCAGCGAGCTGATT
CGTCGTAGCGTGGGCCTGGCGCACGTTGCGGACATCGATACCATTCAGG
ACGATGCGATGCGTCACGAATGCCTGCGTGAAGCGATCACCCTGGGTCG
TGCGCTGATTGTTCTGGCGGAGCGCATCGACAGCGTGGATGAACTGCTG
GCGCGTGTTCGTCAATACAGCCTCGAGCACCACCACCACCACCACTGA

In specific occurrences of this first instance, the MTR kinases described herein include polypeptides having the amino acid sequence as set forth below in SEQ ID NO: 9.

(SEQ ID NO: 9)
MSQYHTFTADDAVAYAQQFAGIDNPSELVSAQEVGDGNLNLVFKVFDRQ
GVSRAIVKQALPYPRAVGPSWPLTLDRARHEAQTLVAHYQHSPQHTVKI
HHFDPELAVMVMEDLSDHRIWRGELIANVYYPQAARQLGDYLAQVLFHT
SDFYLHPHEKKAQVAQFINPGMCEISEDLSENDPYQIHERNNYPAELEA
DVAALRDDAQLKLAVAALKHRFFAHAEALLHGDLHSGSIFVAEGSLKVI
DAEFGYFGPIGFDIGTAIGNLLLNYCGLPGQLGIRDAAAAREQRLNDIH
QLWTTFAERFQALAAEKTRDAALAYPGYASAFLKKVWADAVGFCGSELI
RRSVGLSHVADIDTIQDDAMRHECLRHAITLGRALIVLAETIDSVDELL
ARVRQYSLEHHHHHH

In specific examples of such occurrences, the polypeptides comprising the amino acid sequence as set forth above in SEQ ID NO: 9 may be encoded by the DNA sequence as set forth below in SEQ ID NO: 10.

(SEQ ID NO: 10)
ATGAGCCAGTATCATACCTTCACCGCGGATGATGCGGTGGCGTATGCGC
AGCAATTTGCGGGCATTGATAACCCGAGCGAGCTGGTTAGCGCGCAAGA
AGTTGGTGACGGCAACCTGAACCTGGTGTTCAAGGTTTTTGATCGTCAG
GGTGTGAGCCGTGCGATCGTTAAACAAGCGCTGCCGTACCCCCGTGCGG
TTGGTCCGAGCTGGCCGCTGACCCTGGACCGTGCGCGTCATGAAGCGCA
GACCCTGGTGGCGCACTATCAGCACAGCCCGCAACACACCGTTAAGATC
CACCACTTCGATCCGGAGCTGGCGGTGATGGTTATGGAAGACCTGAGCG
ATCACCGTATTTGGCGTGGTGAGCTGATCGCGAACGTGTACTATCCGCA
GGCGGCGCGTCAACTGGGTGACTACCTGGCGCAGGTTCTGTTCCACACC
AGCGATTTTTATCTGCACCCGCACGAGAAGAAAGCGCAGGTGGCGCAAT
TCATTAACCCGGGCATGTGCGAAATCAGCGAAGACCTGAGCTTTAACGA
TCCGTACCAGATTCACGAACGTAACAACTATCCGGCGGAGCTGGAAGCG
GATGTGGCGGCGCTGCGTGATGATGCGCAACTGAAGCTGGCGGTTGCGG
CGCTGAAACACCGTTTCTTTGCGCATGCGGAGGCGCTGCTGCATGGTGA
CCTTCACAGCGGCAGCATCTTCGTTGCGGAGGGTAGCCTGAAGGTGATC
GACGCGGAATTCGGTTACTTTGGCCCGATCGGTTTTGATATTGGTACCG
CGATCGGCAACCTGCTGCTGAACTATTGCGGTCTGCCGGGTCAACTGGG
TATTCGTGATGCGGCGGCGGCGCGTGAACAGCGTCTGAACGATATCCAC
CAACTGTGGACCACCTTCGCGGAGCGTTTTCAAGCGCTGGCGGCGGAAA
AGACCCGTGACGCGGCGCTGGCGTACCCGGGTTATGCGAGCGCGTTCCT
GAAGAAAGTGTGGGCGGATGCGGTTGGTTTTTGCGGCAGCGAGCTGATT
CGTCGTAGCGTGGGCCTGTCGCACGTTGCGGACATCGATACCATTCAGG
ACGATGCGATGCGTCACGAATGCCTGCGTCACGCGATCACCCTGGGTCG
TGCGCTGATTGTTCTGGCGGAGACCATCGACAGCGTGGATGAACTGCTG
GCGCGTGTTCGTCAATACAGCCTCGAGCACCACCACCACCACCACTGA

In specific occurrences of this first instance, the MTR kinases described herein include polypeptides having the amino acid sequence as set forth below in SEQ ID NO: 11.

(SEQ ID NO: 11)
MSQYHTFTADDAVAYAQQFAGIDNPSELVSAQEVGDGNLNLVFKVEDR
QGVSRAIVKQALPYPRRVGPSWPLTLDRARHEAQTLVAHYQHSPQHTVK
IFHFDPELAVMVMEDLSDHRIWRGELIANVYYPQAARQLGDYLAQVLFH
TSDFYLHPHEKKRQVAQFINPAMCGISEDLVENDPYQIHERNNYPAELE
AQVAALRDDAQLKLAVAALKHRFFAHAEALLHGDLHTGSIFVKEGSLKV
IDAEFGYFGPIGFDIGTAIGNLLLNYCGLPGQLGIRDAAAAREQRLNDI
HQLWTTFAERFQALAAEKTRDAALAYPGYASAFLKKVWADAVGFCGSEL
IRRSVGLAHVADIDTIQDDAMRHECLREAITLGRALIVLAERIDSVDEL
LARVRQYSLEHHHHHH

In specific occurrences of this first instance, the MTR kinases described herein include polypeptides having the amino acid sequence as set forth below in SEQ ID NO: 12.

(SEQ ID NO: 12)
MSQYHTFTADDAVAYAQQFAGIDNPSELVSAQEVGDGNLNLVFKVFDRQ
GVSRSIVKQALPYPRRVGPSWPLTLDRARHEAQTLVAHYQHSPQHTVKI
FHFDPELAVMVMEDLSDHRIWRGELIANVYYPQAARQLGDYLAQVLFHT
SDFYLHPHEKKRQVAQFINPAMCGISEDLVENDPYQIHERNNYPAELEA
QVAALRDDAQLKLAVAALKHRFFAHAEALLHGDLHTGSIFVKEGSLKVI
DAEFGYFGPIGFDIGTAIGNLLLNYCGLPGQLGIRDAAAAREQRLNDIH
QLWTTFAERFQALAAEKTRDAALRYPGYASAFLKKVWADAVGFCGSELI
RRSVGLAHVADIDTIQDDAMRHECLREAITLGRALIVLAERIDSVDELL
ARVRQYSLEHHHHHH

In some embodiments, an improved MTR kinase of the disclosure is a polypeptide based on the amino acid sequences of SEQ ID NO: 1, 3, 5, 7, 9, 11, or 12 and can comprise an amino acid sequence that is at least about 85%, 86%, 87%, 88%, 89%, 90%, 91%, 92%, 93%, 94%, 95%, 96%, 97%, 98%, or 99% identical to the reference sequence of SEQ ID NO: 1, 3, 5, 7, 9, 11, or 12. These differences can be amino acid insertions, deletions, substitutions, or any combinations of such changes. In some occurrences, the amino acid sequence differences can comprise non-conservative, conservative, as well as a combination of non-conservative and conservative amino acid substitutions.

In some embodiments, an improved MTR kinase of the disclosure is a polypeptide encoded by the DNA sequences of SEQ ID NO: 2, 4, 6, 8, or 10 and can comprise a sequence that is at least about 85%, 86%, 87%, 88%, 89%, 90%, 91%, 92%, 93%, 94%, 95%, 96%, 97%, 98%, or 99% identical to the translated reference sequence of SEQ ID NO: 2, 4, 6, 8, or 10. These differences can be amino acid insertions, deletions, substitutions, or any combinations of such changes. In some occurrences, the sequence differences can comprise non-conservative, conservative, as well as a combination of non-conservative and conservative amino acid substitutions.

Additional embodiments provide host cells comprising the polynucleotides and/or expression vectors described herein. The host cells may be E. coli, or they may be a different organism. The host cells can be used for the expression and isolation of the MTR kinases described herein, or, alternatively, they can be used directly for the conversion of the substrate to the stereoisomeric product.

Whether carrying out the method with whole cells, cell extracts or purified MTR kinases, a single MTR kinase may be used or, alternatively, mixtures of two or more MTR kinases may be used.

Embodiments relate to MTR kinase polypeptides capable of selectively preparing alpha-1-phosphate sugars in the synthesis of nucleosides. In embodiments, the MTR kinase polypeptides are capable of the following conversion:

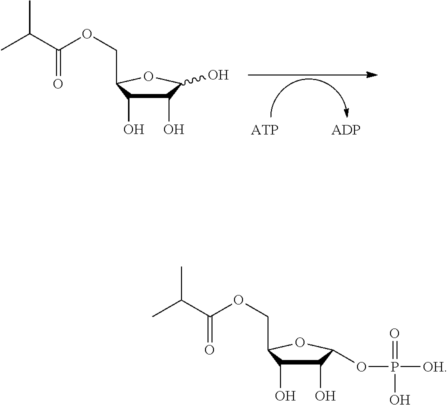

In particular embodiments, the MTR kinase polypeptides are capable of the following conversion:

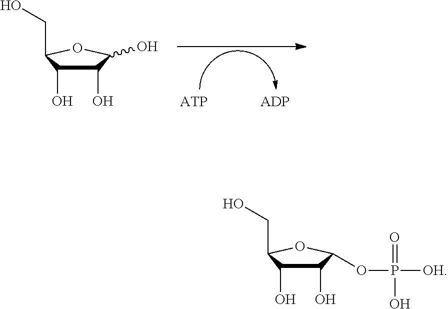

In such embodiments, the alpha-1-phosphate sugars may be in the form of a salt.

Enzyme properties for which improvement is desirable include, but are not limited to, enzymatic activity, thermal stability, pH activity profile, cofactor requirements, refractoriness to inhibitors (e.g., product inhibition), stereospecificity, stereoselectivity, and solvent stability. The improvements can relate to a single enzyme property, such as enzymatic activity, or a combination of different enzyme properties, such as enzymatic activity and stereoselectivity.

Table 1 below provide a list of the SEQ ID NOs disclosed herein with associated activities. The amino acid sequences below are based on the MTR kinase polypeptide sequence of SEQ ID NO: 1, unless otherwise specified. In table below, each row lists a SEQ ID NO. The column listing the number of mutations (i.e., residue changes) refers to the number of amino acid substitutions as compared to the MTR kinase polypeptide sequence of SEQ ID NO: 1. In Table 1, “d.r.” is used to denote “diastereomeric ratio”. The first digit denotes the fraction of the product that is the 5′-isobutyryl-alpha-ribose-1-phosphate form, while the second digit denotes the fraction of the product that is the 5′-isobutyryl-beta-ribose-1-phosphate.

TABLE 1
Enzymatic Activity of MTR Kinase Polypeptides

| | | Number of | | |
|---|---|---|---|---|
| SEQ | Residue Changes | Changes | | |
| ID | Relative to | Relative to | | |
| NO: | SEQ ID NO: 1 | SEQ ID NO: 1 | Conversion | d.r. |
| 1 | — | 0 | 5% | 7:1 |
| 3 | L79H | 1 | 100% | >99:1 |
| 5 | V63P L79H T173S | 5 | 100% | >99:1 |
| | F177I I230L | | | |
| 7 | H10D V63P C65R | 12 | 100% | >99:1 |
| | E68P L79H T173S | | | |
| | F177V I230L S232T | | | |
| | A244V S350A H370E | | | |
| 9 | H10D V63P C65A | 11 | 100% | >99:1 |
| | E68P L79H A168G | | | |
| | T173S F177S I230L | | | |
| | A244V R384T | | | |

### Polynucleotides Encoding MTR Kinases

In another aspect, the present disclosure provides polynucleotides encoding the MTR kinases disclosed herein. The polynucleotides may be operatively linked to one or more heterologous regulatory sequences that control gene expression to create a recombinant polynucleotide capable of expressing the polypeptide. Expression constructs containing a heterologous polynucleotide encoding the MTR kinase can be introduced into appropriate host cells to express the corresponding MTR kinase polypeptide.

Because of the knowledge of the codons corresponding to the various amino acids, availability of a protein sequence provides a description of all the polynucleotides capable of encoding the subject. The degeneracy of the genetic code, where the same amino acids are encoded by alternative or synonymous codons allows an extremely large number of nucleic acids to be made, all of which encode the improved MTR kinases disclosed herein. Thus, having identified a particular amino acid sequence, those skilled in the art could make any number of different nucleic acids by simply modifying the sequence of one or more codons in a way that does not change the amino acid sequence of the protein. In this regard, the present disclosure specifically contemplates each and every possible variation of polynucleotides that could be made by selecting combinations based on the possible codon choices, and all such variations are to be considered specifically disclosed for any polypeptide disclosed herein.

In various embodiments, the codons are preferably selected to fit the host cell in which the protein is being produced. For example, preferred codons used in bacteria are used to express the gene in bacteria; preferred codons used in yeast are used for expression in yeast; and preferred codons used in mammals are used for expression in mammalian cells. By way of example, the polynucleotides of SEQ ID NO: 2 have been codon optimized for expression in Escherichia coli.

In certain embodiments, all codons need not be replaced to optimize the codon usage of the MTR kinases since the natural sequence will comprise preferred codons and because use of preferred codons may not be required for all amino acid residues. Consequently, codon optimized polynucleotides encoding the MTR kinases may contain preferred codons at about 40%, 50%, 60%, 70%, 80%, or greater than 90% of codon positions of the full length coding region.

In various embodiments, an isolated polynucleotide encoding an improved MTR kinase polypeptide may be manipulated in a variety of ways to provide for expression of the polypeptide. Manipulation of the isolated polynucleotide prior to its insertion into a vector may be desirable or necessary depending on the expression vector. The techniques for modifying polynucleotides and nucleic acid sequences utilizing recombinant DNA methods are well known in the art. Guidance is provided in Sambrook et al., 2001, MOLECULAR CLONING: A LABORATORY MANUAL, 3rd Ed., Cold Spring Harbor Laboratory Press; and CURRENT PROTOCOLS IN MOLECULAR BIOLOGY, Ausubel. F. ed., Greene Pub. Associates, 1998, updates to 2006.

In some embodiments, an isolated polynucleotide encoding any of the MTR kinase polypeptides herein is manipulated in a variety of ways to facilitate expression of the MTR kinase polypeptide. In some embodiments, the polynucleotides encoding the MTR kinase polypeptides comprise expression vectors where one or more control sequences is present to regulate the expression of the MTR kinase polypeptides. Manipulation of the isolated polynucleotide prior to its insertion into a vector may be desirable or necessary depending on the expression vector utilized. Techniques for modifying polynucleotides and nucleic acid sequences utilizing recombinant DNA methods are well known in the art. In some embodiments, the control sequences include among others, promoters, leader sequences, polyadenylation sequences, propeptide sequences, signal peptide sequences, and transcription terminators. In some embodiments, suitable promoters are selected based on the host cell selection. For bacterial host cells, suitable promoters for directing transcription of the nucleic acid constructs of the present disclosure, include, but are not limited to, promoters obtained from the E. coli lac operon, a hybrid promoter consisting of the T7 RNA polymerase binding site followed by the lac operator sequence. In addition, suitable promotors may include Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (see e.g., Villa-Kamaroff et al., PROC. NATL ACAD. Sci. USA 75: 3727-3731 (1978)), as well as the tac promoter (see e.g., DeBoer et al., PROC. NATL ACAD. Sci. USA 80: 21-25 (1983)).

In some embodiments, the control sequence is also a suitable transcription terminator sequence (i.e., a sequence recognized by a host cell to terminate transcription). In some embodiments, the terminator sequence is operably linked to the 3′ terminus of the nucleic acid sequence encoding the enzyme polypeptide. Any suitable terminator that is functional in the host cell of choice finds use in the present disclosure. An exemplary transcription terminator for Escherichia coli includes the T7 terminator.

In some embodiments, the control sequence is also a suitable leader sequence (i.e., a non-translated region of an mRNA that is important for translation by the host cell). In some embodiments, the leader sequence is operably linked to the 5′ terminus of the nucleic acid sequence encoding the MTR kinase. Any suitable leader sequence that is functional in the host cell of choice find use in the present disclosure. Exemplary leaders for E. coli will encode a ribosome binding site.

In some embodiments, regulatory sequences are also utilized. These sequences facilitate the regulation of the expression of the polypeptide relative to the growth of the host cell. Examples of regulatory systems are those that cause the expression of the gene to be turned on or off in response to a chemical or physical stimulus, including the presence of a regulatory compound. In prokaryotic host cells, suitable regulatory sequences include, but are not limited to, the lac, tac, and trp operator systems. In yeast host cells, suitable regulatory systems include, but are not limited to, the ADH2 system or GAL1 system. In filamentous fungi, suitable regulatory sequences include, but are not limited to, the TAKA alpha-amylase promoter, Aspergillus niger glucoamylase promoter, and Aspergillus oryzae glucoamylase promoter.

In another aspect, the present disclosure is directed to a recombinant expression vector comprising a polynucleotide encoding MTR kinase polypeptide, and one or more expression regulating regions such as a promoter and a terminator, a replication origin, etc., depending on the type of hosts into which they are to be introduced. In some embodiments, the various nucleic acid and control sequences described herein are joined together to produce recombinant expression vectors that include one or more convenient restriction sites to allow for insertion or substitution of the nucleic acid sequence encoding the enzyme polypeptide at such sites. Alternatively, in some embodiments, the nucleic acid sequence of the present disclosure is expressed by inserting the nucleic acid sequence or a nucleic acid construct comprising the sequence into an appropriate vector for expression. In some embodiments involving the creation of the expression vector, the coding sequence is located in the vector so that the coding sequence is operably linked with the appropriate control sequences for expression.

The recombinant expression vector may be any suitable vector (e.g., a plasmid or virus), that can be conveniently subjected to recombinant DNA procedures and bring about the expression of the enzyme polynucleotide sequence. The choice of the vector typically depends on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector is one in which, when introduced into the host cell, it is integrated into the genome and replicated together with the chromosome(s) into which it has been integrated. Furthermore, in some embodiments, a single vector or plasmid, or two or more vectors or plasmids which together contain the total DNA to be introduced into the genome of the host cell, and/or a transposon is utilized.

In some embodiments, the expression vector contains one or more selectable markers, which permit easy selection of transformed cells. A “selectable marker” is a gene, the product of which provides for biocide or viral resistance, resistance to heavy metals, prototrophy to auxotrophs, and the like. Examples of bacterial selectable markers include, but are not limited to, the dal genes from Bacillus subtilis or Bacillus licheniformis, or markers, which confer antibiotic resistance such as ampicillin, kanamycin, chloramphenicol or tetracycline resistance. Suitable markers for yeast host cells include, but are not limited to, ADE2, HIS3, LEU2, LYS2, MET3, TRP1, and URA3. Selectable markers for use in filamentous fungal host cells include, but are not limited to, amdS (acetamidase; e.g., from A. nidulans or A. orzyae), argB (ornithine carbamoyltransferases), bar (phosphinothricin acetyltransferase; e.g., from S. hygroscopicus), hph (hygromycin phosphotransferase), niaD (nitrate reductase), pyrG (orotidine-5′-phosphate decarboxylase; e.g., from A. nidulans or A. orzyae), sC (sulfate adenyltransferase), and trpC (anthranilate synthase), as well as equivalents thereof.

In another aspect, the present disclosure provides a host cell comprising at least one polynucleotide encoding at least one MTR kinase of the present disclosure, the polynucleotide(s) being operatively linked to one or more control sequences for expression of the at least one MTR kinase in the host cell. Host cells suitable for use in expressing the polypeptides encoded by the expression vectors of the present disclosure are well known in the art and include but are not limited to, bacterial cells, such as E. coli, Vibrio fluvialis, Streptomyces and Salmonella typhimurium cells; fungal cells, such as yeast cells (e.g., Saccharomyces cerevisiae or Pichia pastoris (ATCC Accession No. 201178)). Exemplary host cells also include various Escherichia coli strains (e.g., W3110 (ΔfhuA) and BL21). Examples of bacterial selectable markers include, but are not limited to, the dal genes from Bacillus subtilis or Bacillus licheniformis, or markers, which confer antibiotic resistance such as ampicillin, kanamycin, chloramphenicol, and or tetracycline resistance.

In some alternative embodiments, the expression vectors contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements preferably contain a sufficient number of nucleotides, such as 100 to 10,000 base pairs, preferably 400 to 10,000 base pairs, and most preferably 800 to 10,000 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are P15A ori or the origins of replication of plasmids pBR322, pUC19, pACYC177 (which contains the P15A ori), or pACYC184 (which contains the P15A ori) permitting replication in E. coli, and pUB110, pE194, or pTA1060 permitting replication in Bacillus. Examples of origins of replication for use in a yeast host cell are the 2 micron origin of replication, ARS1, ARS4, the combination of ARS1 and CEN3, and the combination of ARS4 and CEN6. The origin of replication may be one having a mutation which makes its functioning temperature-sensitive in the host cell (see e.g., Ehrlich, Proc. Natl. Acad. Sci. USA 75:1433 (1978)).

In some embodiments, more than one copy of a nucleic acid sequence of the present disclosure is inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

Many of the expression vectors for use in the present disclosure are commercially available. Suitable commercial expression vectors include, but are not limited to, Novagen™'s pET E. coli T7 expression vectors (Millipore Sigma) and the p3xFLAG™ expression vectors (Sigma-Aldrich Chemicals). Other suitable expression vectors include, but are not limited to, pBluescriptII SK(-) and pBK-CMV (Stratagene), and plasmids derived from pBR322 (Gibco BRL), pUC (Gibco BRL), pREP4, pCEP4 (Invitrogen) or pPoly (see e.g., Lathe et al., Gene 57:193-201 (1987)).

Thus, in some embodiments, a vector comprising a sequence encoding at least one variant MTR kinase is transformed into a host cell in order to allow propagation of the vector and expression of the variant MTR kinase(s). In some embodiments, the transformed host cell described above is cultured in a suitable nutrient medium under conditions permitting the expression of the variant MTR kinase(s). Any suitable medium useful for culturing the host cells finds use in the present disclosure, including, but not limited to minimal or complex media containing appropriate supplements. In some embodiments, host cells are grown in HTP media. Suitable media are available from various commercial suppliers or may be prepared according to published recipes (e.g., in catalogues of the American Type Culture Collection).

### Host Cells for Expression of MTR Kinases

In another aspect, the present disclosure provides a host cell comprising a polynucleotide encoding an improved MTR kinase polypeptide of the present disclosure, the polynucleotide being operatively linked to one or more control sequences for expression of the MTR kinase in the host cell. Host cells for use in expressing the MTR kinase polypeptides encoded by the expression vectors of the present disclosure are well known in the art and include but are not limited to, bacterial cells, such as Klebsiella aerogenes, Klebsiella pneumoniae, E. coli, B. subtilis, B. licheniformis, B. megaterium, B. stearothermophilus, B. amyloliquefaciens, Lactobacillus kejir, Lactobacillus brevis, Lactobacillus minor, Streptomyces and Salmonella typhimurium cells; fungal cells, such as yeast cells (e.g., Saccharomyces cerevisiae or Pichia pastoris (ATCC Accession No. 201178)). Appropriate culture mediums and growth conditions for the above-described host cells are well known in the art.

Polynucleotides for expression of the MTR kinases may be introduced into cells by various methods known in the art. Techniques include among others, electroporation, biolistic particle bombardment, liposome mediated transfection, calcium chloride transfection, and protoplast fusion. Various methods for introducing polynucleotides into cells will be apparent to the skilled artisan.

In some embodiments of the present disclosure, the filamentous fungal host cells are of any suitable genus and species, including, but not limited to Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora, Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Trametes, Tolypocladium, Trichoderma, Verticillium, and/or Volvariella, and/or teleomorphs, or anamorphs, and synonyms, basionyms, or taxonomic equivalents thereof.

In some embodiments of the present disclosure, the host cell is a yeast cell, including but not limited to cells of Candida, Hansenula, Saccharomyces, Schizosaccharomyces, Pichia, Kluyveromyces, or Yarrowia species. In some embodiments of the present disclosure, the yeast cell is Hansenula polymorpha, Saccharomyces cerevisiae, Saccharomyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, or Yarrowia lipolytica.

In some other embodiments, the host cell is a prokaryotic cell. Suitable prokaryotic cells include, but are not limited to, Gram-positive, Gram-negative and Gram-variable bacterial cells. Any suitable bacterial organism finds use in the present disclosure, including but not limited to Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia and Zymomonas. In some embodiments, the host cell is a species of Agrobacterium, Acinetobacter, Azobacter, Bacillus, Bifidobacterium, Buchnera, Geobacillus, Campylobacter, Clostridium, Corynebacterium, Escherichia, Enterococcus, Erwinia, Flavobacterium, Lactobacillus, Lactococcus, Pantoea, Pseudomonas, Staphylococcus, Salmonella, Streptococcus, Streptomyces, or Zymomonas. In some embodiments, the bacterial host strain is non-pathogenic to humans. In some embodiments the bacterial host strain is an industrial strain. Numerous bacterial industrial strains are known and suitable in the present disclosure. In some embodiments of the present disclosure, the bacterial host cell is an Agrobacterium species (e.g., A. radiobacter, A. rhizogenes, and A. rubi). In some embodiments of the present disclosure, the bacterial host cell is an Arthrobacter species (e.g., A. aurescens, A. citreus, A. globiformis, A. hydrocarboglutamicus, A. mysorens, A. nicotianae, A. paraffineus, A. protophonniae, A. roseoparqffinus, A. sulfureus, and A. ureafaciens). In some embodiments of the present disclosure, the bacterial host cell is a Bacillus species (e.g., B. thuringensis, B. anthracis, B. megaterium, B. subtilis, B. lentus, B. circulans, B. pumilus, B. lautus, B. coagulans, B. brevis, B. firmus, B. alkaophius, B. licheniformis, B. clausii, B. stearothermophilus, B. halodurans, and B. amyloliquefaciens). In some embodiments, the host cell is an industrial Bacillus strain including but not limited to B. subtilis, B. pumilus, B. licheniformis, B. megaterium, B. clausii, B. stearothermophilus, or B. amyloliquefaciens. In some embodiments, the Bacillus host cells are B. subtilis, B. licheniformis, B. megaterium, B. stearothermophilus, and/or B. amyloliquefaciens. In some embodiments, the bacterial host cell is a Clostridium species (e.g., C. acetobutylicum, C. tetani E88, C. lituseburense, C. saccharobutylicum, C. perfringens, and C. beijerinckii). In some embodiments, the bacterial host cell is a Corynebacterium species (e.g., C. glutamicum and C. acetoacidophilum). In some embodiments the bacterial host cell is an Escherichia species (e.g., E. coli). In some embodiments, the host cell is Escherichia coli W3110. In some embodiments the host is Escherichia coli BL21 or BL21(DE3). In some embodiments, the bacterial host cell is an Erwinia species (e.g., E. uredovora, E. carotovora, E. ananas, E. herbicola, E. punctata, and E. terreus). In some embodiments, the bacterial host cell is a Pantoea species (e.g., P. citrea, and P. agglomerans). In some embodiments the bacterial host cell is a Pseudomonas species (e.g., P. putida, P. aeruginosa, P. mevalonii, and P. sp. D-01 10). In some embodiments, the bacterial host cell is a Streptococcus species (e.g., S. equisimiles, S. pyogenes, and S. uberis). In some embodiments, the bacterial host cell is a Streptomyces species (e.g., S. ambofaciens, S. achromogenes, S. avermitilis, S. coelicolor, S. aureofaciens, S. aureus, S. fungicidicus, S. griseus, and S. lividans). In some embodiments, the bacterial host cell is a Zymomonas species (e.g., Z. mobilis, and Z. lipolytica).

Many prokaryotic and eukaryotic strains that find use in the present disclosure are readily available to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

In some embodiments, host cells are genetically modified to have characteristics that improve protein secretion, protein stability and/or other properties desirable for expression and/or secretion of a protein. Genetic modification can be achieved by genetic engineering techniques and/or classical microbiological techniques (e.g., chemical or UV mutagenesis and subsequent selection). Indeed, in some embodiments, combinations of recombinant modification and classical selection techniques are used to produce the host cells. Using recombinant technology, nucleic acid molecules can be introduced, deleted, inhibited or modified, in a manner that results in increased yields of MTR kinase variant(s) within the host cell and/or in the culture medium. In one genetic engineering approach, homologous recombination is used to induce targeted gene modifications by specifically targeting a gene in vivo to suppress expression of the encoded protein. In alternative approaches, siRNA, antisense and/or ribozyme technology find use in inhibiting gene expression. A variety of methods are known in the art for reducing expression of protein in cells, including, but not limited to deletion of all or part of the gene encoding the protein and site-specific mutagenesis to disrupt expression or activity of the gene product (see e.g., Chaveroche et al., NUCL. ACIDS RES., 28:22 e97 (2000); Cho et al., MOLEC. PLANT MICROBE INTERACT., 19:7-15 (2006); Maruyama and Kitamoto, BIOTECHNOL LETT., 30:1811-1817 (2008); Takahashi et al., MOL. GEN. GENOM., 272: 344-352 (2004); and You et al., Arch. Microbiol.,191:615-622 (2009), all of which are incorporated by reference herein). Random mutagenesis, followed by screening for desired mutations also finds use (see e.g., Combier et al., FEMS MICROBIOL. LETT., 220:141-8 (2003); and Firon et al., EUKARY. CELL 2:247-55 (2003), both of which are incorporated by reference).

Introduction of a vector or DNA construct into a host cell can be accomplished using any suitable method known in the art, including but not limited to calcium phosphate transfection, DEAE-dextran mediated transfection, PEG-mediated transformation, electroporation, or other common techniques known in the art.

In some embodiments, the engineered host cells (i.e., “recombinant host cells”) of the present disclosure are cultured in conventional nutrient media modified as appropriate for activating promoters, selecting transformants, or amplifying the MTR kinase polynucleotide. Culture conditions, such as temperature, pH and the like, are those previously used with the host cell selected for expression, and are well-known to those skilled in the art. As noted, many standard references and texts are available for the culture and production of many cells, including cells of bacterial, plant, animal (especially mammalian) and archebacterial origin.

In some embodiments, cells expressing the MTR kinase of the disclosure are grown under batch or continuous fermentations conditions. Classical “batch fermentation” is a closed system, wherein the compositions of the medium are set at the beginning of the fermentation and is not subject to artificial alternations during the fermentation. A variation of the batch system is a “fed-batch fermentation” that also finds use in the present disclosure. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art. “Continuous fermentation” is an open system where a defined fermentation medium is added continuously to a bioreactor and an equal amount of conditioned medium is removed simultaneously for processing. Continuous fermentation generally maintains the cultures at a constant high density where cells are primarily in log phase growth. Continuous fermentation systems strive to maintain steady state growth conditions. Methods for modulating nutrients and growth factors for continuous fermentation processes as well as techniques for maximizing the rate of product formation are well known in the art of industrial microbiology.

More than one copy of a nucleic acid sequence of the present disclosure may be inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

In some embodiments of the present disclosure, cell-free transcription and translation systems find use in producing the MTR kinase(s). Several systems are commercially available, and the methods are well-known to those skilled in the art.

### Methods of Evolving MTR Kinases

In some embodiments, to make the MTR kinases of the present disclosure, the MTR kinase that catalyzes 1-phosphorylation of ribose and 5-isobutyrylribose is obtained (or derived) from Escherichia coli. In some embodiments, the parent polynucleotide sequence is codon optimized to enhance expression of the MTR kinase in a specified host cell. The parental polynucleotide sequence, designated as SEQ ID NO: 2, was codon optimized for expression in Escherichia coli, and the codon-optimized polynucleotide cloned into an expression vector, placing the expression of the MTR kinase gene under the control of the tac promoter. The T7 RNA polymerase needed to express the gene of interest is under control of the lacUV5 promoter, and both the gene of interest and the T7 RNA polymerase are subject to repression by glucose. The presence of lactose or lactose analogs such as isopropyl β-d-1-thiogalactopyranoside (IPTG) activates the T7 RNA polymerase production and eliminates the repression, resulting in production of the MTR kinase gene. Clones expressing the active MTR kinase in Escherichia coli were identified, and the genes sequenced to confirm their identity.

The MTR kinases of the disclosure may be obtained by subjecting the polynucleotide encoding the parent sequence to mutagenesis and/or directed evolution methods. An exemplary directed evolution technique is mutagenesis and/or DNA shuffling as described in Stemmer, 1994, PROC. NATL. ACAD. Sci. USA 91:10747-10751; WO 95/22625; WO 97/20078; WO 97/35966; WO 98/27230; WO 00/42651; WO 01/75767 and U.S. Pat. No. 6,537,746. Other directed evolution procedures that can be used include, among others, staggered extension process (StEP), in vitro recombination (Zhao et al., 1998, NAT. BIOTECHNOL. 16:258-261), mutagenic PCR (Caldwell et al., 1994, PCR METHODS APPL. 3:S136-S140), and cassette mutagenesis (Black et al., 1996, PROC. NATL. ACAD. Sci. USA 93:3525-3529).

The clones obtained following mutagenesis treatment are screened for MTR kinases having a desired improved enzyme property. Measuring enzyme activity from the expression libraries can be performed using standard chemistry analytical techniques for measuring substrates and products such as UPLC-MS. In this reaction, the gamma phosphate residue of ATP is transferred to the substrate at the 1-position by the MTR kinase to yield ADP and the phosphorylated ribose or 5-isobutyrylribose. The reaction may also be run under conditions where the MTR kinase is the yield-limiting catalyst, such that a doubling or halving in concentration of the MTR kinase will cause a doubling or halving of the yield of nucleoside product observed at a given timepoint. Where the improved enzyme property desired is thermal stability, enzyme activity may be measured after subjecting the enzyme preparations to a defined temperature and measuring the amount of enzyme activity remaining after heat treatments. Clones containing a polynucleotide encoding an MTR kinase are then isolated, sequenced to identify the nucleotide sequence changes (if any), and used to express the enzyme in a host cell.

Where the sequence of the polypeptide is known, the polynucleotides encoding the enzyme can be prepared by standard solid-phase methods, according to known synthetic methods. In some embodiments, fragments of up to about 100 bases can be individually synthesized, then joined (e.g., by enzymatic or chemical litigation methods, or polymerase mediated methods) to form any desired continuous sequence. For example, polynucleotides and oligonucleotides of the disclosure can be prepared by chemical synthesis using, e.g., the classical phosphoramidite method described by Beaucage et al., 1981, TET. LETT. 22:1859-69, or the method described by Matthes et al., 1984, EMBO J. 3:801-05, e.g., as it is typically practiced in automated synthetic methods. According to the phosphoramidite method, oligonucleotides are synthesized, e.g., in an automatic DNA synthesizer, purified, annealed, ligated and cloned in appropriate vectors. In addition, essentially any nucleic acid can be obtained from any of a variety of commercial sources, such as The Midland Certified Reagent Company, Midland, Tex., The Great American Gene Company, Ramona, Calif, ExpressGen Inc. Chicago, Ill., Operon Technologies Inc., Alameda, Calif, and many others.

MTR kinase polypeptides expressed in a host cell can be recovered from the cells and or the culture medium using any one or more of the well-known techniques for protein purification, including, among others, lysozyme treatment, sonication, filtration, salting-out, ultra-centrifugation, and chromatography. Suitable solutions for lysing and the high efficiency extraction of proteins from bacteria, such as Escherichia coli, are commercially available under the trade name B-PER™ from ThermoFisher Scientific.

Chromatographic techniques for isolation of the MTR kinase polypeptide include, among others, reverse phase chromatography high performance liquid chromatography, ion exchange chromatography, gel electrophoresis, and affinity chromatography. Conditions for purifying a particular enzyme will depend, in part, on factors such as net charge, hydrophobicity, hydrophilicity, molecular weight, molecular shape, etc., and will be apparent to those having skill in the art.

In some embodiments, affinity techniques may be used to isolate the improved MTR kinase polypeptides. For affinity chromatography purification, the protein sequence can be tagged with a recognition sequence to enable purification. Common tags include celluose-binding domains, poly His-tags, di-His chelates, FLAG-tags and many others that will be apparent to those having skill in the art. Antibodies can also be used as affinity purification reagents. Any antibody that specifically binds the MTR kinase polypeptide may be used.

### Methods of Using the MTR Kinases

The MTR kinases described herein can catalyze the phosphorylation of substrate compounds of Formula A:

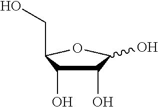

to the corresponding isomeric product of Formula B:

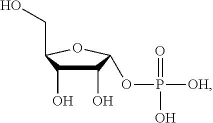

or a salt thereof.
In particular embodiments, the MTR kinases described herein can catalyze the phosphorylation of the substrate compound of Formula C:

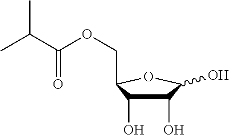

to the corresponding isomeric product of Formula D:

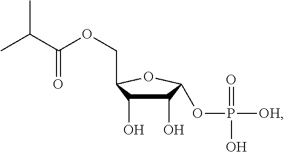

or a salt thereof.

In some embodiments, the method for phosphorylating comprises contacting or incubating the substrate with an MTR kinase disclosed herein under reaction conditions suitable for phosphorylating. In some embodiments, the product in greater than about 99%, 99.1%, 99.2%, 99.3%, 99.4%, 99.5%, 99.6%, 99.7%, 99.8%, or 99.9% diastereometric excess over the corresponding minor product.

In some embodiments of the method for phosphorylating the substrate to the product, the substrate is phosphorylated to give the product in greater than about 85% diastereometric excess, wherein the MTR kinase polypeptide comprises a sequence that corresponds to SEQ ID NO: 1, 3, 5, 7, 9, 11, or 12. In some embodiments of the method for phosphorylating the substrate to the product, the substrate is phosphorylated to provide the product in greater than about 85% diastereometric excess, wherein the MTR kinase polypeptide is encoded by a DNA sequence that corresponds to SEQ ID NO: 2, 4, 6, 8, or 10.

In another embodiment of this method for phosphorylating the substrate to obtain the product, at least about 95% of the substrate is converted to obtain the product in less than about 24 hours when carried out with greater than about 50 g/L of substrate and less than about 5 g/L of the polypeptide, wherein the polypeptide comprises an amino acid sequence corresponding to SEQ ID NO: 1, 3, 5, 7, 9, 11, or 12. In embodiments of the method for phosphorylating the substrate to obtain the product, at least about 95% of the substrate is converted to the product in less than about 24 hours when carried out with greater than about 50 g/L of substrate and less than about 5 g/L of the polypeptide, wherein the MTR kinase polypeptide is encoded by a DNA sequence that corresponds to SEQ ID NO: 2, 4, 6, 8, or 10.

In some embodiments, the method for phosphorylating comprises a process for preparing a phosphorylated ribose or phosphate ribose derivative, or a salt thereof, said process comprising reacting ribose or a ribose derivative with a phosphate source in the presence of a polypeptide comprising an amino acid sequence corresponding to SEQ ID NO: 1, 3, 5, 7, 9, 11, or 12, or wherein the MTR kinase polypeptide is encoded by a DNA sequence that corresponds to SEQ ID NO: 2, 4, 6, 8, or 10. In specific embodiments, the ribose or ribose derivative is selected from the group consisting of

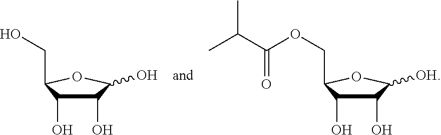

In particular embodiments, the process comprises reacting ribose with a phosphate source in the presence of a polypeptide comprising an amino acid sequence corresponding to SEQ ID NO: 1, 3, 5, 7, 9, 11, or 12, or a polypeptide that is encoded by a DNA sequence that corresponds to SEQ ID NO: 2, 4, 6, 8, or 10:

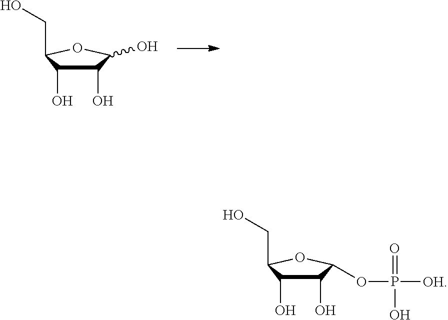

In other particular embodiments, the process comprises reacting ribose with a phosphate source in the presence of a polypeptide comprising an amino acid sequence corresponding to SEQ ID NO: 1, 3, 5, 7, 9, 11, or 12, or a polypeptide that is encoded by a DNA sequence that corresponds to SEQ ID NO: 2, 4, 6, 8, or 10:

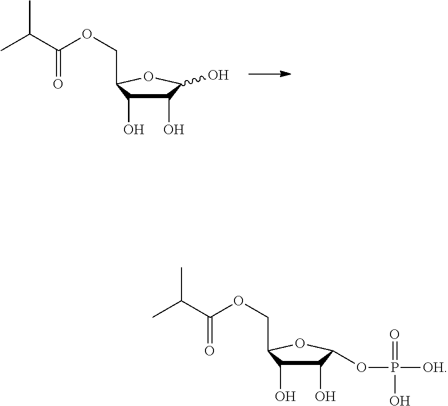

Additional embodiments of the disclosure provide a compound selected from the group consisting of

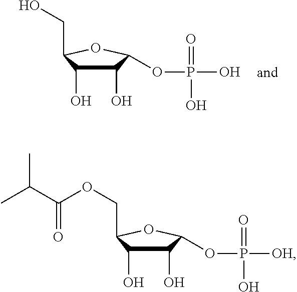

and salts thereof.

As is known by those of skill in the art, kinase catalyzed reactions typically require a cofactor. Reactions catalyzed by the MTR kinase polypeptides described herein also typically require a cofactor, although many embodiments of the engineered MTR kinases require far less cofactor than reactions catalyzed with wild-type MTR kinases. As used herein, the term “cofactor” refers to a non-protein compound that operates in combination with an MTR kinase enzyme. Cofactors suitable for use with the engineered MTR kinases described herein include, but are not limited to, divalent cations, including Mg²⁺, Ca²⁺, Mn²⁺, or Co²⁺, and nucleotide triphosphates, including ATP, which act as cofactors that transfer a phosphate residue to the substrate.

The MTR kinase-catalyzed phosphorylation reactions described herein are generally carried out in a solvent. In some embodiments, aqueous solvents, including water and aqueous co-solvent systems, are used.

Exemplary aqueous co-solvent systems have water and one or more organic solvent. In general, an organic solvent component of an aqueous co-solvent system is selected such that it does not completely inactivate the MTR kinase enzyme. Appropriate co-solvent systems can be readily identified by measuring the enzymatic activity of the specified engineered MTR kinase with a defined substrate of interest in the candidate solvent system, utilizing an enzyme activity assay, such as those described herein.

The organic solvent component of an aqueous co-solvent system may be miscible with the aqueous component, providing a single liquid phase, or may be partly miscible or immiscible with the aqueous component, providing two liquid phases. Generally, when an aqueous co-solvent system is employed, it is selected to be biphasic, with water dispersed in an organic solvent, or vice-versa. Generally, when an aqueous co-solvent system is utilized, it is desirable to select an organic solvent that can be readily separated from the aqueous phase. In general, the ratio of water to organic solvent in the co-solvent system is typically in the range of from about 99:1 to about 10:90 (v/v) organic solvent to water, and between 80:20 and 20:80 (v/v) organic solvent to water. The co-solvent system may be pre-formed prior to addition to the reaction mixture, or it may be formed in situ in the reaction vessel.

The aqueous solvent (water or aqueous co-solvent system) may be pH-buffered or unbuffered. Generally, the phosphorylation can be carried out at a pH in the range of from about 5 to about 9. In some embodiments, the phosphorylation is carried out at a pH of about 8 or below, often in the range of from about 5 to about 8, and usually in the range of from about 6.5 to about 8.

During the course of the phosphorylation reactions, the pH of the reaction mixture may change. The pH of the reaction mixture may be maintained at a desired pH or within a desired pH range by the addition of an acid or a base during the course of the reaction. Alternatively, the pH may be controlled by using an aqueous solvent that comprises a buffer. Suitable buffers to maintain desired pH ranges are known in the art and include, for example, phosphate buffer, triethanolamine buffer, and the like. Combinations of buffering and acid or base addition may also be used.

When the stoichiometric acetyl or propionyl phosphate ATP recycling is employed, the co-production of acetic or propionic acid (pKa=3.6), as represented in equation (1) causes the pH of the reaction mixture to drop if the resulting aqueous acetic or propionic acid (pKa=4.7) is not otherwise neutralized. The pH of the reaction mixture may be maintained at the desired level by standard buffering techniques, wherein the buffer neutralizes the acetic acid or propionic acid up to the buffering capacity provided, or by the addition of a base concurrent with the course of the conversion. Combinations of buffering and base addition may also be used. Suitable buffers to maintain desired pH ranges are described above. Suitable bases for neutralization of acetic acid or propionic acid are organic bases, for example amines, alkoxides and the like, and inorganic bases, for example, hydroxide salts (e.g., NaOH), carbonate salts (e.g., NaHCO₃), bicarbonate salts (e.g., K₂CO₃), basic phosphate salts (e.g., K₂HPO₄, Na₃PO₄), and the like. The addition of a base concurrent with the course of the conversion may be done manually while monitoring the reaction mixture pH or, more conveniently, by using an automatic titrator as a pH stat. A combination of partial buffering capacity and base addition can also be used for process control.

When base addition is employed to neutralize acetic acid or propionic acid released during an MTR kinase-catalyzed phosphorylation reaction, the progress of the conversion may be monitored by the amount of base added to maintain the pH. Typically, bases added to unbuffered or partially buffered reaction mixtures over the course of the phosphorylation are added in aqueous solutions.

In some embodiments, when the process is carried out using whole cells of the host organism, the whole cell may natively provide ATP. Alternatively or in combination, the cell may natively or recombinantly provide the pyruvate oxidase or other enzymes required to recycle ATP.

In carrying out the stereoselective phosphorylation reactions described herein, the MTR kinase, and any enzymes comprising the optional ATP regeneration system, may be added to the reaction mixture in the form of the purified enzymes, whole cells transformed with gene(s) encoding the enzymes, and/or cell extracts and/or lysates of such cells. The gene(s) encoding the MTR kinase polypeptide and the optional ATP regeneration enzymes can be transformed into host cells separately or together into the same host cell. For example, in some embodiments one set of host cells can be transformed with gene(s) encoding the MTR kinase and another set can be transformed with gene(s) encoding the ATP regeneration enzymes. Both sets of transformed cells can be utilized together in the reaction mixture in the form of whole cells, or in the form of lysates or extracts derived therefrom. In other embodiments, a host cell can be transformed with gene(s) encoding both the MTR kinase polypeptide and the ATP regeneration enzymes.

Whole cells transformed with gene(s) encoding the MTR kinase polypeptide and/or the optional ATP regeneration enzymes, or cell extracts and/or lysates thereof, may be employed in a variety of different forms, including solid (e.g., lyophilized, spray-dried, and the like) or semisolid (e.g., a crude paste).

The cell extracts or cell lysates may be partially purified by precipitation (ammonium sulfate, polyethyleneimine, heat treatment, or the like), followed by a desalting procedure prior to lyophilization (e.g., ultrafiltration, dialysis, and the like). Any of the cell preparations may be stabilized by crosslinking using known crosslinking agents.

Suitable conditions for carrying out the MTR kinase catalyzed reactions described herein include a wide variety of conditions that can be readily optimized by routine experimentation that includes, but is not limited to, contacting the engineered MTR kinase polypeptide and substrate at an experimental pH and temperature and detecting product, for example, using the methods described in the Examples provided herein.

The MTR kinase catalyzed phosphorylation is typically carried out at a temperature in the range of from about 20° C. to about 55° C. In embodiments, it is carried out at a temperature in the range of from about 20° C. to about 45° C. The reaction may also be carried out under ambient conditions.

The reaction is generally allowed to proceed until essentially complete, or near complete, phosphorylation of substrate is obtained. Phosphorylation of substrate to product can be monitored using known methods by detecting substrate and/or product. Suitable methods include HPLC-CAD (charged aerosol detection), HPLC-MS, and the like. Conversion yields of the phosphorylation product generated in the reaction mixture are generally greater than about 50%, may also be greater than about 60%, may also be greater than about 70%, may also be greater than about 80%, may also be greater than 90%, and are often greater than about 97%.

### EXAMPLES

### Abbreviations

ATP adenosine 5′-triphosphate
DNaseI Commercially available endonuclease that nonspecifically cleaves DNA to release di-, tri- and oligonucleotide products with 5′-phosphorylated and 3′-hydroxylated ends
IPTG Isopropyl β-D-1-thiogalactopyranoside
LB-agar Luria-Bertani medium, commercially available, nutritionally rich medium, for culture and growth of bacteria
LB-broth Luria-Bertani broth, commercially available, nutritionally rich medium, for culture and growth of bacteria
NdeI Commercially available restriction enzyme that is an endonuclease isolated from Neisseria denarificans
pET30 Commercially available system for expression of recombinant proteins in E. coli
XhoI Commercially available restriction enzyme
ZYM-5052 Commercially available, nutritionally rich medium for culture and growth of bacteria
w/v Weight per volume

### Example 1: Enzyme Preparation for Well Plate Reactions

A codon-optimized wild-type MTR kinase from Klebsiella pneumoniae was synthesized and cloned into pET30 via NdeI/XhoI restriction sites. The plasmid containing the wild-type MTR kinase from K. pneumoniae was transformed into electrocompetent Escherichia coli BL21(DE3) via electroporation. Following a 1 hour outgrowth at 37° C., a portion of the transformation was plated on LB-agar supplemented with 50 micrograms per mL kanamycin and 1% (w/v) glucose. The following day, colonies were picked and inoculated into a 96-well plate containing 0.2 mL per well of Luria-Bretani Broth (culture media for cells) supplemented with 50 micrograms per mL of kanamycin and 1% (w/v) glucose. The 96-well plate was shaken at 250 RPM/30° C. overnight. The following day, 0.01 mL per well of culture from each well was used to inoculate the corresponding well of a new 96-well plate containing 0.39 mL per well of ZYM-5052 medium. This culture was grown at 30° C./250 RPM for 18 hours after which time the cells were pelleted by centrifugation and the supernatants were discarded. After centrifugation, cells were frozen, thawed, and then resuspended in lysis buffer (0.2 mL per well of 50 mM triethanolamine-HCl pH 7.5, 1 mg/mL lysozyme, 0.5 mg/mL polymyxin B sulfate, 1 U/mL DNaseI, and 1 mM MgSO₄). The plate was then shaken for 2 h at room temperature at 1200 RPM. The lysate was then clarified by centrifugation (4000×g, 10 minutes). The supernatant following this step was then used in subsequent well-plate reactions (Example 3).

### Example 2: Enzyme Preparation for Vial and Larger-Scale Reactions

A codon-optimized wild-type MTR kinase from K. pneumoniae was synthesized and cloned into pET30 via NdeI/XhoI restriction sites and ligated using T4 ligase. The plasmid was sequence verified using T7f and T7TRM sequencing primers. The plasmid containing the wild-type MTR kinase from K. pneumoniae was transformed into electrocompetent Escherichia coli BL21(DE3) via electroporation. Following a 1 hour outgrowth at 37° C., a portion of the transformation was plated on LB-agar supplemented with 50 micrograms per mL kanamycin and 1% (w/v) glucose. The following day, a single colony was picked and inoculated into a 125 mL flask containing 50 mL of Luria-Bretani Broth (culture media for cells) supplemented with 50 micrograms per mL of kanamycin and 1% (w/v) glucose. The flask was shaken at 250 RPM/30° C. overnight. The following day, a 2.8 L flask containing 1 L of ZYM-5052 medium was inoculated with a 100-fold dilution of the saturated overnight culture. This culture was grown at 30° C./250 RPM for 18 hours after which time the cells were harvested by centrifugation. After centrifugation, cells were resuspended to a concentration of 0.33 grams of 1 mL of aqueous 50 mM triethanolamine-HCl buffer pH 7.5. This suspension was shaken at 20° C. for 30 minutes, after which time the cells were placed on ice to chill, and then disrupted by high-pressure homogenization (16,000 PSI). The resulting lysate was then clarified by centrifuging at 10,000×g for 30 minutes. Following centrifugation, the supernatant was frozen and lyophilized.

### Example 3: MTR Kinase Reaction in Well Plates

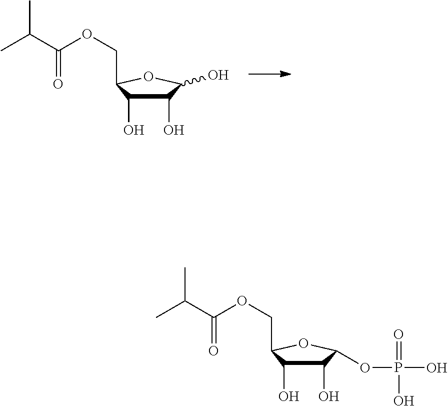

To a well of a 96-well round-bottom polypropylene plate was charged 5′-isobutyryl ribose (1.14M in water, 16 μL, 4 mg), propionyl phosphate monoammonium salt (1M in water, pH adjusted to 7.5, 25 μL, 4.3 mg), triethanolamine (1M in water, pH adjusted to 7.5, 40 μL, 6.0 mg), magnesium chloride (0.1M in water, 10 μL, 0.095 mg), ATP (0.1M in water, 2 μL, 0.11 mg), acetate kinase with the amino acid sequence specified by SEQ ID NO: 13 below (20 g/L in water, 4 μL ,0.08 mg), S-methylthioribose kinase with the amino acid sequence specified by SEQ ID NO. 3 (2 g/L in water, 100 μL, 0.2 mg). The plate was sealed and shaken at 25° C., 700 RPM overnight. 10 μL of the reaction mixture was taken and diluted with 190 μL 50/50 (v/v) acetonitrile:water for UPLC-MS analysis. 95% assay yield was obtained.

(SEQ ID NO: 13)
MGSHHHHHHGSRVLNINSGSSSIKYQLIEMEGEKVLCKGIAERIGIEGS
RLVHRVGDEKHVIERELPDHEEALKLILNTLVDEKLGVIKDLKEIDAVG
HRVVHGGERFKESVLVDEEVLKAIEEVSPLAPLHNPANLMGIKAAMKLL
PGVPNVQVEDTAFHQTIPQKAYLYAIPYEYYEKYKIRRYGFHGISHRYV
SKRAAEILGKKLEELKIITCHIGNGASVAAVKYGKCVDTSMGFTPLEGL
VMGTRSGDLDPAIPFFIMEKEGISPQEMYDILNKKSGVYGLSKGFSSDM
RDNFEAALKGDEWCKLVLEIYDYRIAKYIGAYAAAMNGVDAIVFTAGVG
ENSPITREDVCKYLEFLGVKLDKQKNEETILGKEGIISTPDSRVKVLVV
PTNEELMIARDTKEIVEKIGR

### Example 4: MTR Kinase Reaction in Vials

Into a clean and dry Falcon tube added 60 mg triethanolamine, 4 mg of MgCl, hexahydrate, 200 mg of proprionyl phosphate monoammonium salt, and 3 mg of ATP, along with 4 mL water. The pH of the solution was around 4.0, so adjustment was made with 6M KOH solution to 7.5. Then 200 mg of 5′-isobutyrylribose and 4 mg of acetate kinase with the amino acid sequence specified by SEQ ID NO: 13 above were added to the pH adjusted stock solution. pH did not change.

To two clean and dry 2 dram vials, added 50 mg of MTR kinase polypeptide variants followed by 1 mL stock solution of other reagents. The reactions were stirred at 30° C. and at 500 rpm, until judged complete. Table 2 shows the conversions.

TABLE 2

| Vial | Enzyme SEQ ID NO | pH | % Conversion |
|---|---|---|---|
| A | 1 | 7.5 | <5% |
| B | 3 | 6.5 | >99% |

It will be appreciated that various of the above-discussed and other features and functions, or alternatives thereof, may be desirably combined into many other different systems or applications. Also that various alternatives, modifications, variations or improvements therein may be subsequently made by those skilled in the art that are also intended to be encompassed by the following claims.

1. A polypeptide, wherein said polypeptide comprises a region with an amino acid sequence having at least a 90% sequence identity to residues of any one of SEQ ID NO: 3, SEQ ID NO: 5, SEQ ID NO: 7, SEQ ID NO: 9, SEQ ID NO: 11, or SEQ ID NO: 12.

2. The polypeptide according to claim 1, wherein said polypeptide comprises a region with an amino acid sequence having at least a 90% sequence identity to residues of SEQ ID NO: 3.

3. The polypeptide according to claim 1, wherein said polypeptide comprises a region with an amino acid sequence having at least a 90% sequence identity to residues of SEQ ID NO: 5.

4. The polypeptide according to claim 1, wherein said polypeptide comprises a region with an amino acid sequence having at least a 90% sequence identity to residues of SEQ ID NO: 7.

5. The polypeptide according to claim 1, wherein said polypeptide comprises a region with an amino acid sequence having at least a 90% sequence identity to residues of SEQ ID NO: 9.

6. The polypeptide according to claim 1, wherein said polypeptide comprises a region with an amino acid sequence having at least a 90% sequence identity to residues of SEQ ID NO: 11.

7. The polypeptide according to claim 1, wherein said polypeptide comprises a region with an amino acid sequence having at least a 90% sequence identity to residues of SEQ ID NO: 12.

8. A polypeptide, wherein said polypeptide is encoded by a DNA sequence having at least 90% sequence identity to residues of any one of SEQ ID NO: 4, SEQ ID NO: 6, SEQ ID NO: 8, or SEQ ID NO: 10.

9. The polypeptide according to claim 8, wherein said polypeptide is encoded by a DNA sequence having at least a 90% sequence identity to residues of SEQ ID NO: 4.

10. The polypeptide according to claim 8, wherein said polypeptide is encoded by a DNA sequence having at least a 90% sequence identity to residues of SEQ ID NO: 6.

11. The polypeptide according to claim 8, wherein said polypeptide is encoded by a DNA sequence having at least a 90% sequence identity to residues of SEQ ID NO: 8.

12. The polypeptide according to claim 8, wherein said polypeptide is encoded by a DNA sequence having at least a 90% sequence identity to residues of SEQ ID NO: 10.

13. An expression vector comprising the polypeptide according to claim 1, operably linked to one or more control sequences suitable for directing expression of the encoded polypeptide in a host cell.

14. The expression vector of claim 13, wherein the control sequence comprises a promoter.

15. The expression vector of claim 14, wherein the promoter comprises an E. coli promoter.

16. A host cell comprising the expression vector of claim 13.

17. The host cell of claim 16, wherein said host cell is E. coli.

18. A process for preparing a phosphorylated ribose or phosphorylated ribose derivative, or a salt thereof, said process comprising reacting ribose or a ribose derivative with a phosphate source in the presence of a polypeptide according to claim 1.

19. The process of claim 18, wherein the ribose or ribose derivative is selected from the group consisting of

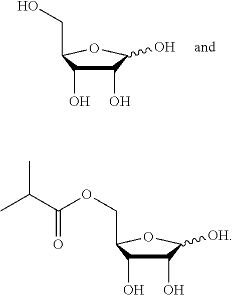

20. The process of claim 18, wherein the process comprises reacting ribose with a phosphate source in the presence of the polypeptide:

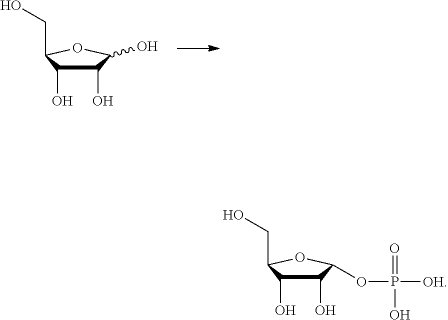

21. The process of claim 18, wherein the process comprises reacting ribose with a phosphate source in the presence of the polypeptide:

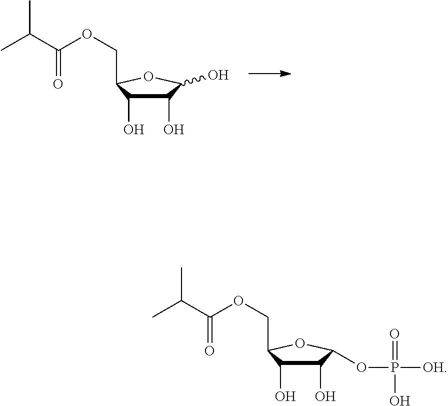

22. A compound selected from the group consisting of

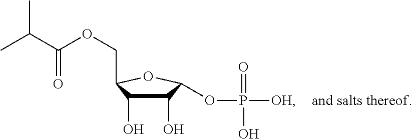

23. The compound of claim 22, wherein the compound is [chemical structure]


SEQUENCE LISTING (13 sequences)

SEQ ID NO: 1 - protein, 399 aa, Klebsiella pneumoniae
MSQYHTFTAHDAVAYAQQFAGIDNPSELVSAQEVGDGNLNLVFKVFDRQGVSRAIVKQALPYVRCVGESWPLTLDRARLEAQTLVAHYQHSPQHTVKIHHFDPELAVMVMEDLSDHRIWRGELIANVYYPQAARQLGDYLAQVLFHTSDFYLHPHEKKAQVAQFINPAMCEITEDLFFNDPYQIHERNNYPAELEADVAALRDDAQLKLAVAALKHRFFAHAEALLHGDIHSGSIFVAEGSLKAIDAEFGYFGPIGFDIGTAIGNLLLNYCGLPGQLGIRDAAAAREQRLNDIHQLWTTFAERFQALAAEKTRDAALAYPGYASAFLKKVWADAVGFCGSELIRRSVGLSHVADIDTIQDDAMRHECLRHAITLGRALIVLAERIDSVDELLARVRQYS

SEQ ID NO: 2 - DNA, 1197 bp, Artificial Sequence
  Description of Artificial Sequence Synthetic polynucleotide
  codon optimized sequence encoding wild-type polypeptide
atgagccagtatcataccttcaccgcgcatgatgcggtggcgtatgcgcagcaatttgcgggcattgataacccgagcgagctggttagcgcgcaagaagttggtgacggcaacctgaacctggtgttcaaggtttttgatcgtcagggtgtgagccgtgcgatcgttaaacaagcgctgccgtacgtgcgttgcgttggtgaaagctggccgctgaccctggaccgtgcgcgtctggaagcgcagaccctggtggcgcactatcagcacagcccgcaacacaccgttaagatccaccacttcgatccggagctggcggtgatggttatggaagacctgagcgatcaccgtatttggcgtggtgagctgatcgcgaacgtgtactatccgcaggcggcgcgtcaactgggtgactacctggcgcaggttctgttccacaccagcgatttttatctgcacccgcacgagaagaaagcgcaggtggcgcaattcattaacccggcgatgtgcgagatcaccgaagacctgttctttaacgatccgtaccagattcacgaacgtaacaactatccggcggagctggaagcggatgtggcggcgctgcgtgatgatgcgcaactgaagctggcggttgcggcgctgaaacaccgtttctttgcgcatgcggaggcgctgctgcatggtgacattcacagcggcagcatcttcgttgcggagggtagcctgaaggcgatcgacgcggaattcggttactttggcccgatcggttttgatattggtaccgcgatcggcaacctgctgctgaactattgcggtctgccgggtcaactgggtattcgtgatgcggcggcggcgcgtgaacagcgtctgaacgatatccaccaactgtggaccaccttcgcggagcgttttcaagcgctggcggcggaaaagacccgtgacgcggcgctggcgtacccgggttatgcgagcgcgttcctgaagaaagtgtgggcggatgcggttggtttttgcggcagcgagctgattcgtcgtagcgtgggcctgagccacgttgcggacatcgataccattcaggacgatgcgatgcgtcacgaatgcctgcgtcacgcgatcaccctgggtcgtgcgctgattgttctggcggagcgtatcgacagcgtggatgaactgctggcgcgtgttcgtcaatacagc

SEQ ID NO: 3 - protein, 407 aa, Artificial Sequence
  Description of Artificial Sequence Synthetic polypeptide
  MTR kinase AA seq
MSQYHTFTAHDAVAYAQQFAGIDNPSELVSAQEVGDGNLNLVFKVFDRQGVSRAIVKQALPYVRCVGESWPLTLDRARHEAQTLVAHYQHSPQHTVKIHHFDPELAVMVMEDLSDHRIWRGELIANVYYPQAARQLGDYLAQVLFHTSDFYLHPHEKKAQVAQFINPAMCEITEDLFFNDPYQIHERNNYPAELEADVAALRDDAQLKLAVAALKHRFFAHAEALLHGDIHSGSIFVAEGSLKAIDAEFGYFGPIGFDIGTAIGNLLLNYCGLPGQLGIRDAAAAREQRLNDIHQLWTTFAERFQALAAEKTRDAALAYPGYASAFLKKVWADAVGFCGSELIRRSVGLSHVADIDTIQDDAMRHECLRHAITLGRALIVLAERIDSVDELLARVRQYSLEHHHHHH

SEQ ID NO: 4 - DNA, 1224 bp, Artificial Sequence
  Description of Artificial Sequence Synthetic polynucleotide
  MTR kinase DNA seq
atgagccagtatcataccttcaccgcgcatgatgcggtggcgtatgcgcagcaatttgcgggcattgataacccgagcgagctggttagcgcgcaagaagttggtgacggcaacctgaacctggtgttcaaggtttttgatcgtcagggtgtgagccgtgcgatcgttaaacaagcgctgccgtacgtgcgttgcgttggtgaaagctggccgctgaccctggaccgtgcgcgtcatgaagcgcagaccctggtggcgcactatcagcacagcccgcaacacaccgttaagatccaccacttcgatccggagctggcggtgatggttatggaagacctgagcgatcaccgtatttggcgtggtgagctgatcgcgaacgtgtactatccgcaggcggcgcgtcaactgggtgactacctggcgcaggttctgttccacaccagcgatttttatctgcacccgcacgagaagaaagcgcaggtggcgcaattcattaacccggcgatgtgcgagatcaccgaagacctgttctttaacgatccgtaccagattcacgaacgtaacaactatccggcggagctggaagcggatgtggcggcgctgcgtgatgatgcgcaactgaagctggcggttgcggcgctgaaacaccgtttctttgcgcatgcggaggcgctgctgcatggtgacattcacagcggcagcatcttcgttgcggagggtagcctgaaggcgatcgacgcggaattcggttactttggcccgatcggttttgatattggtaccgcgatcggcaacctgctgctgaactattgcggtctgccgggtcaactgggtattcgtgatgcggcggcggcgcgtgaacagcgtctgaacgatatccaccaactgtggaccaccttcgcggagcgttttcaagcgctggcggcggaaaagacccgtgacgcggcgctggcgtacccgggttatgcgagcgcgttcctgaagaaagtgtgggcggatgcggttggtttttgcggcagcgagctgattcgtcgtagcgtgggcctgagccacgttgcggacatcgataccattcaggacgatgcgatgcgtcacgaatgcctgcgtcacgcgatcaccctgggtcgtgcgctgattgttctggcggagcgtatcgacagcgtggatgaactgctggcgcgtgttcgtcaatacagcctcgagcaccaccaccaccaccactga

SEQ ID NO: 5 - protein, 407 aa, Artificial Sequence
  Description of Artificial Sequence Synthetic polypeptide
  MTR kinase AA seq
MSQYHTFTAHDAVAYAQQFAGIDNPSELVSAQEVGDGNLNLVFKVFDRQGVSRAIVKQALPYPRCVGESWPLTLDRARHEAQTLVAHYQHSPQHTVKIHHFDPELAVMVMEDLSDHRIWRGELIANVYYPQAARQLGDYLAQVLFHTSDFYLHPHEKKAQVAQFINPAMCEISEDLIFNDPYQIHERNNYPAELEADVAALRDDAQLKLAVAALKHRFFAHAEALLHGDLHSGSIFVAEGSLKAIDAEFGYFGPIGFDIGTAIGNLLLNYCGLPGQLGIRDAAAAREQRLNDIHQLWTTFAERFQALAAEKTRDAALAYPGYASAFLKKVWADAVGFCGSELIRRSVGLSHVADIDTIQDDAMRHECLRHAITLGRALIVLAERIDSVDELLARVRQYSLEHHHHHH

SEQ ID NO: 6 - DNA, 1224 bp, Artificial Sequence
  Description of Artificial Sequence Synthetic polynucleotide
  MTR kinase DNA seq
atgagccagtatcataccttcaccgcgcatgatgcggtggcgtatgcgcagcaatttgcgggcattgataacccgagcgagctggttagcgcgcaagaagttggtgacggcaacctgaacctggtgttcaaggtttttgatcgtcagggtgtgagccgtgcgatcgttaaacaagcgctgccgtacccccgttgcgttggtgaaagctggccgctgaccctggaccgtgcgcgtcatgaagcgcagaccctggtggcgcactatcagcacagcccgcaacacaccgttaagatccaccacttcgatccggagctggcggtgatggttatggaagacctgagcgatcaccgtatttggcgtggtgagctgatcgcgaacgtgtactatccgcaggcggcgcgtcaactgggtgactacctggcgcaggttctgttccacaccagcgatttttatctgcacccgcacgagaagaaagcgcaggtggcgcaattcattaacccggcgatgtgcgagatcagcgaagacctgatttttaacgatccgtaccagattcacgaacgtaacaactatccggcggagctggaagcggatgtggcggcgctgcgtgatgatgcgcaactgaagctggcggttgcggcgctgaaacaccgtttctttgcgcatgcggaggcgctgctgcatggtgaccttcacagcggcagcatcttcgttgcggagggtagcctgaaggcgatcgacgcggaattcggttactttggcccgatcggttttgatattggtaccgcgatcggcaacctgctgctgaactattgcggtctgccgggtcaactgggtattcgtgatgcggcggcggcgcgtgaacagcgtctgaacgatatccaccaactgtggaccaccttcgcggagcgttttcaagcgctggcggcggaaaagacccgtgacgcggcgctggcgtacccgggttatgcgagcgcgttcctgaagaaagtgtgggcggatgcggttggtttttgcggcagcgagctgattcgtcgtagcgtgggcctgagccacgttgcggacatcgataccattcaggacgatgcgatgcgtcacgaatgcctgcgtcacgcgatcaccctgggtcgtgcgctgattgttctggcggagcgtatcgacagcgtggatgaactgctggcgcgtgttcgtcaatacagcctcgagcaccaccaccaccaccactga

SEQ ID NO: 7 - protein, 407 aa, Artificial Sequence
  Description of Artificial Sequence Synthetic polypeptide
  MTR kinase AA seq
MSQYHTFTADDAVAYAQQFAGIDNPSELVSAQEVGDGNLNLVFKVFDRQGVSRAIVKQALPYPRRVGPSWPLTLDRARHEAQTLVAHYQHSPQHTVKIHHFDPELAVMVMEDLSDHRIWRGELIANVYYPQAARQLGDYLAQVLFHTSDFYLHPHEKKAQVAQFINPAMCEISEDLVFNDPYQIHERNNYPAELEADVAALRDDAQLKLAVAALKHRFFAHAEALLHGDLHTGSIFVAEGSLKVIDAEFGYFGPIGFDIGTAIGNLLLNYCGLPGQLGIRDAAAAREQRLNDIHQLWTTFAERFQALAAEKTRDAALAYPGYASAFLKKVWADAVGFCGSELIRRSVGLAHVADIDTIQDDAMRHECLREAITLGRALIVLAERIDSVDELLARVRQYSLEHHHHHH

SEQ ID NO: 8 - DNA, 1224 bp, Artificial Sequence
  Description of Artificial Sequence Synthetic polynucleotide
  MTR kinase DNA seq
atgagccagtatcataccttcaccgcggatgatgcggtggcgtatgcgcagcaatttgcgggcattgataacccgagcgagctggttagcgcgcaagaagttggtgacggcaacctgaacctggtgttcaaggtttttgatcgtcagggtgtgagccgtgcgatcgttaaacaagcgctgccgtacccccgtcgcgttggtccgagctggccgctgaccctggaccgtgcccgtcatgaagcgcagaccctggttgcgcactatcagcacagcccgcaacacaccgttaagatccaccacttcgatccggagctggcggtgatggttatggaagacctgagcgatcaccgtatttggcgtggtgagctgatcgcgaacgtgtactatccgcaggcggcgcgtcaactgggtgactacctggcgcaggttctgttccacaccagcgatttttatctgcacccgcacgagaagaaagcgcaggtggcgcaattcattaacccggccatgtgcgaaatcagcgaagacctggtgtttaacgatccgtaccagattcacgaacgtaacaactatccggcggagctggaagcggatgtggcggcgctgcgtgatgatgcgcaactgaagctggcggttgcggcgctgaaacaccgtttctttgcgcatgcggaggcgctgctgcatggtgaccttcacaccggcagcatcttcgttgcggagggtagcctgaaggtgatcgacgcggaattcggttactttggcccgatcggttttgatattggtaccgcgatcggcaacctgctgctgaactattgcggtctgccgggtcaactgggtattcgtgatgcggcggcggcgcgtgaacagcgtctgaacgatatccaccaactgtggaccaccttcgcggagcgttttcaagcgctggcggcggaaaagacccgtgacgcggcgctggcgtacccgggttatgcgagcgcgttcctgaagaaagtgtgggcggatgcggttggtttttgcggcagcgagctgattcgtcgtagcgtgggcctggcgcacgttgcggacatcgataccattcaggacgatgcgatgcgtcacgaatgcctgcgtgaagcgatcaccctgggtcgtgcgctgattgttctggcggagcgcatcgacagcgtggatgaactgctggcgcgtgttcgtcaatacagcctcgagcaccaccaccaccaccactga

SEQ ID NO: 9 - protein, 407 aa, Artificial Sequence
  Description of Artificial Sequence Synthetic polypeptide
  MTR kinase AA seq
MSQYHTFTADDAVAYAQQFAGIDNPSELVSAQEVGDGNLNLVFKVFDRQGVSRAIVKQALPYPRAVGPSWPLTLDRARHEAQTLVAHYQHSPQHTVKIHHFDPELAVMVMEDLSDHRIWRGELIANVYYPQAARQLGDYLAQVLFHTSDFYLHPHEKKAQVAQFINPGMCEISEDLSFNDPYQIHERNNYPAELEADVAALRDDAQLKLAVAALKHRFFAHAEALLHGDLHSGSIFVAEGSLKVIDAEFGYFGPIGFDIGTAIGNLLLNYCGLPGQLGIRDAAAAREQRLNDIHQLWTTFAERFQALAAEKTRDAALAYPGYASAFLKKVWADAVGFCGSELIRRSVGLSHVADIDTIQDDAMRHECLRHAITLGRALIVLAETIDSVDELLARVRQYSLEHHHHHH

SEQ ID NO: 10 - DNA, 1224 bp, Artificial Sequence
  Description of Artificial Sequence Synthetic polynucleotide
  MTR kinase DNA seq
atgagccagtatcataccttcaccgcggatgatgcggtggcgtatgcgcagcaatttgcgggcattgataacccgagcgagctggttagcgcgcaagaagttggtgacggcaacctgaacctggtgttcaaggtttttgatcgtcagggtgtgagccgtgcgatcgttaaacaagcgctgccgtacccccgtgcggttggtccgagctggccgctgaccctggaccgtgcgcgtcatgaagcgcagaccctggtggcgcactatcagcacagcccgcaacacaccgttaagatccaccacttcgatccggagctggcggtgatggttatggaagacctgagcgatcaccgtatttggcgtggtgagctgatcgcgaacgtgtactatccgcaggcggcgcgtcaactgggtgactacctggcgcaggttctgttccacaccagcgatttttatctgcacccgcacgagaagaaagcgcaggtggcgcaattcattaacccgggcatgtgcgaaatcagcgaagacctgagctttaacgatccgtaccagattcacgaacgtaacaactatccggcggagctggaagcggatgtggcggcgctgcgtgatgatgcgcaactgaagctggcggttgcggcgctgaaacaccgtttctttgcgcatgcggaggcgctgctgcatggtgaccttcacagcggcagcatcttcgttgcggagggtagcctgaaggtgatcgacgcggaattcggttactttggcccgatcggttttgatattggtaccgcgatcggcaacctgctgctgaactattgcggtctgccgggtcaactgggtattcgtgatgcggcggcggcgcgtgaacagcgtctgaacgatatccaccaactgtggaccaccttcgcggagcgttttcaagcgctggcggcggaaaagacccgtgacgcggcgctggcgtacccgggttatgcgagcgcgttcctgaagaaagtgtgggcggatgcggttggtttttgcggcagcgagctgattcgtcgtagcgtgggcctgtcgcacgttgcggacatcgataccattcaggacgatgcgatgcgtcacgaatgcctgcgtcacgcgatcaccctgggtcgtgcgctgattgttctggcggagaccatcgacagcgtggatgaactgctggcgcgtgttcgtcaatacagcctcgagcaccaccaccaccaccactga

SEQ ID NO: 11 - protein, 407 aa, Artificial Sequence
  Description of Artificial Sequence Synthetic polypeptide
  MTR kinase AA seq
MSQYHTFTADDAVAYAQQFAGIDNPSELVSAQEVGDGNLNLVFKVFDRQGVSRAIVKQALPYPRRVGPSWPLTLDRARHEAQTLVAHYQHSPQHTVKIFHFDPELAVMVMEDLSDHRIWRGELIANVYYPQAARQLGDYLAQVLFHTSDFYLHPHEKKRQVAQFINPAMCGISEDLVFNDPYQIHERNNYPAELEAQVAALRDDAQLKLAVAALKHRFFAHAEALLHGDLHTGSIFVKEGSLKVIDAEFGYFGPIGFDIGTAIGNLLLNYCGLPGQLGIRDAAAAREQRLNDIHQLWTTFAERFQALAAEKTRDAALAYPGYASAFLKKVWADAVGFCGSELIRRSVGLAHVADIDTIQDDAMRHECLREAITLGRALIVLAERIDSVDELLARVRQYSLEHHHHHH

SEQ ID NO: 12 - protein, 407 aa, Artificial Sequence
  Description of Artificial Sequence Synthetic polypeptide
  MTR kinase AA seq
MSQYHTFTADDAVAYAQQFAGIDNPSELVSAQEVGDGNLNLVFKVFDRQGVSRSIVKQALPYPRRVGPSWPLTLDRARHEAQTLVAHYQHSPQHTVKIFHFDPELAVMVMEDLSDHRIWRGELIANVYYPQAARQLGDYLAQVLFHTSDFYLHPHEKKRQVAQFINPAMCGISEDLVFNDPYQIHERNNYPAELEAQVAALRDDAQLKLAVAALKHRFFAHAEALLHGDLHTGSIFVKEGSLKVIDAEFGYFGPIGFDIGTAIGNLLLNYCGLPGQLGIRDAAAAREQRLNDIHQLWTTFAERFQALAAEKTRDAALRYPGYASAFLKKVWADAVGFCGSELIRRSVGLAHVADIDTIQDDAMRHECLREAITLGRALIVLAERIDSVDELLARVRQYSLEHHHHHH

SEQ ID NO: 13 - protein, 413 aa, Artificial Sequence
  Description of Artificial Sequence Synthetic polypeptide
  Acetate kinase
MGSHHHHHHGSRVLNINSGSSSIKYQLIEMEGEKVLCKGIAERIGIEGSRLVHRVGDEKHVIERELPDHEEALKLILNTLVDEKLGVIKDLKEIDAVGHRVVHGGERFKESVLVDEEVLKAIEEVSPLAPLHNPANLMGIKAAMKLLPGVPNVQVFDTAFHQTIPQKAYLYAIPYEYYEKYKIRRYGFHGISHRYVSKRAAEILGKKLEELKIITCHIGNGASVAAVKYGKCVDTSMGFTPLEGLVMGTRSGDLDPAIPFFIMEKEGISPQEMYDILNKKSGVYGLSKGFSSDMRDNFEAALKGDEWCKLVLEIYDYRIAKYIGAYAAAMNGVDAIVFTAGVGENSPITREDVCKYLEFLGVKLDKQKNEETILGKEGIISTPDSRVKVLVVPTNEELMIARDTKEIVEKIGR